(19) United States
(12) Patent Application Publication
(10) Pub. No.: US 2021/0403547 A1
(43) Pub. Date: Dec. 30, 2021

(54) Antibodies that bind TGF-Alpha and Epiregulin for use in the treatment of pain

(71) Applicant: Eli Lilly and Company, Indianapolis, IN (US)

(72) Inventors: Marcio Chedid, Fishers, IN (US); Josef G. Heuer, Carmel, IN (US)

(21) Appl. No.: 17/354,195

(22) Filed: Jun. 22, 2021

Related U.S. Application Data
(60) Provisional application No. 63/070,507, filed on Aug. 26, 2020.
(60) Provisional application No. 63/044,455, filed on Jun. 26, 2020.

(51) Int. Cl. C07K 16/22; A61P 43/00

(52) U.S. Cl. CPC C07K 16/22; A61K 2039/505; A61P 43/00

(57) ABSTRACT

The present invention provides for therapeutic use of antibodies that bind human TGF-alpha and human Epiregulin in the treatment of chronic pain, and in particular chronic osteoarthritis pain, chronic diabetic neuropathy pain, or chronic low back pain.

The present invention relates to uses of antibodies that bind human TGF-alpha and Epiregulin in the treatment of chronic pain, including nociceptive, neuropathic, and mixed pain, and in particular in the treatment of chronic osteoarthritis (OA) pain, or chronic diabetic peripheral neuropathy pain (DPNP), or chronic low back pain.

Chronic pain is divided into different categories based on the mechanism: nociceptive, neuropathic, and mixed. Nociceptive pain is caused by stimuli that potentially or actually cause an injury to non-neuronal tissues. This activates nociceptive receptors in the peripheral sensory system. Pain due to osteoarthritis (OA) is a classic example of somatic nociceptive pain. Neuropathic pain is caused by injuries to or disease of the central or peripheral nervous system, leading to maladaptive hypersensitivity of the sensory nervous system. Pain due to diabetic peripheral neuropathy (DPNP) is a classic example of peripheral neuropathic pain. Conditions that exhibit features of both nociceptive and neuropathic pain, such as chronic low back pain, are categorized as mixed pain.

Chronic pain is a highly prevalent condition with huge societal impact. In 2016, an estimated 20.4% of the adult population in the United States experienced chronic pain, defined as pain on most days, or every day in the past 6 months, based on data from the National Health Interview Survey. An estimated 8% of the population had chronic pain that limited their lives or work activities on most days or every day in the past 6 months. As a result, chronic pain is a leading cause for health care expenditure, with the annual cost for managing chronic pain in the United States in 2010 estimated at approximately $635 billion. Despite the high disease burden and societal impact, management of chronic pain is currently unsatisfactory. Nonpharmacologic therapy alone is seldom adequate for pain relief or functional improvement, and available pharmacologic therapies offer modest benefit and have significant safety risks. Presently, the most frequently used drugs to alleviate the most common types of chronic pain are acetaminophen, nonsteroidal anti-inflammatory drugs, and opioids. Gabapentinoids, other anticonvulsants (such as sodium divalproate, carbamazepine, or lamotrigine) and some antidepressants (such as tricyclics or duloxetine) can be used for some specific pain disorders. The current pharmacologic armamentarium typically shows low levels of efficacy, tolerability issues, and/or deleterious side effects. Opioids are effective against acute pain, but they are a limited treatment option for chronic pain because of high abuse risk and potentially serious adverse reactions. The physical, emotional, and financial impact of chronic pain on the patient and society, combined with a lack of efficacious and tolerable treatment options, makes it a significant unmet medical need.

Data suggest that the EGFR pathway is involved in the pathogenesis of neuropathic pain (Kersten, C, Cameron M G, Laird B, Mjåland S. Epidermal growth factor receptor-inhibition (EGFR-I) in the treatment of neuropathic pain. Br J Anaesth. 2015; 115(5):761-767). However, targeting the receptor with an EGFR antibody or EGFR tyrosine kinase inhibitors is associated with high incidence of gastrointestinal (GI) and skin adverse reactions that limit their potential use in the non-oncologic population with chronic pain. Ligands that bind and activate EGFR include epiregulin (EREG), transforming growth factor α (TGFα), epidermal growth factor, heparin-binding epidermal-like growth factor, betacellulin, amphiregulin and epigen (Schneider M R, Wolf E. The epidermal growth factor receptor ligands at a glance. J Cell Physiol. 2009; 218(3):460-466). Two of the EGFR ligands, TGFα and epiregulin, are unique in that they fail to induce receptor degradation and thus promote receptor recycling and persistent EGFR pathway activation (Roepstorff K, Grandal M V, Henriksen L, Knudesen S L, Lerdrup M, Grøvdal L, Willumsen B M, van Deurs B. Differential effects of EGFR ligands on endocytic sorting of the receptor. Traffic. 2009; 10(8):1115-1127).

Antibody I is a high-affinity humanized immunoglobulin G4 (IgG4) monoclonal antibody that binds to key residues in the C-terminal regions of human TGFα and epiregulin, preventing their binding to and activation of EGFR, and Antibody I, and methods of preparing this antibody and formulations thereof are disclosed in WO 2012/138510, along with methods of treatment of diabetic nephropathy.

There remains unmet need for alternative and or improved treatments for chronic pain, including nociceptive, neuropathic, and mixed pain, and in particular in the treatment of osteoarthritis (OA), or diabetic peripheral neuropathy (DPNP), or chronic low back pain. Further there remains unmet need for alternative and or improved treatments for chronic pain, including nociceptive, neuropathic, and mixed pain, and in particular in the treatment of osteoarthritis (OA), or diabetic peripheral neuropathy (DPNP), or chronic low back pain to treat therapy resistant pain which is defined herein as pain refractory to two or more prior monotherapy and/or dual therapy treatment regimens.

The present invention provides antibodies against TGF-alpha and Epiregulin for the treatment of chronic pain, including nociceptive, neuropathic, and mixed pain, and in particular treatment of chronic osteoarthritis (OA) pain, or chronic diabetic peripheral neuropathy pain (DPNP), or chronic low back pain. Furthermore, the present invention provides antibodies against TGF-alpha and Epiregulin for the treatment of chronic osteoarthritis (OA) pain, or chronic diabetic peripheral neuropathy pain (DPNP), or chronic low back pain refractory to two or more prior monotherapy and/or dual therapy treatment regimens.

In an embodiment the present invention provides a method of treating chronic pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

In an embodiment the present invention provides a method of treating chronic osteoarthritis pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

In an embodiment the present invention provides a method of treating chronic diabetic peripheral neuropathy pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

In an embodiment the present invention provides a method of treating chronic low back pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

In an embodiment the present invention provides the method according to any one of embodiments above, wherein the amino acid sequence of the LCVR is SEQ ID NO:9 or SEQ ID NO:10.

In an embodiment the present invention provides the method according to any one of the embodiments above, wherein the amino acid sequence of the HCVR is SEQ ID NO:7.

In an embodiment the present invention provides the method according to any one of the embodiments above, wherein the amino acid sequence of the LCVR is SEQ ID NO:9 and the amino acid sequence of the HCVR is SEQ ID NO:7.

In an embodiment the present invention provides the method according to any one of the embodiments above, wherein the amino acid sequence of the light chain is SEQ ID NO:13 or SEQ ID NO:14.

In an embodiment the present invention provides the method according to any one of the embodiments above, wherein the amino acid sequence of the heavy chain is SEQ ID NO:12.

In an embodiment the present invention provides the method according to any one of the embodiments above, comprising two light chains wherein the amino acid sequence of each light chain is SEQ ID NO:13, and two heavy chains wherein the amino acid sequence of each heavy chain is SEQ ID NO:12.

In an embodiment the present invention provides the method according to any one of the embodiments above, comprising two light chains wherein the amino acid sequence of each light chain is SEQ ID NO:14, and two heavy chains wherein the amino acid sequence of each heavy chain is SEQ ID NO:12.

In an embodiment the present invention provides the method according to any one of the embodiments above, wherein the dose of antibody is a 750 mg starting dose, followed by a 500 mg dose every 2 weeks, for as long as the patient needs treatment for pain.

In an embodiment the present invention provides the method according to any one of the embodiments above wherein the chronic pain is refractory to two or more prior monotherapy and/or dual therapy treatment regimens.

In an embodiment the present invention provides an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3, for use in the treatment of chronic pain. In an embodiment the present invention further provides the above use wherein the chronic pain is selected from the group consisting of chronic osteoarthritis pain, chronic diabetic neuropathy pain, and chronic low back pain. In an embodiment the present invention further provides the above use, wherein the amino acid sequence of the LCVR is SEQ ID NO:9 or SEQ ID NO:10. In an embodiment the present invention further provides the above use wherein the amino acid sequence of the HCVR is SEQ ID NO:7. In an embodiment the present invention further provides the above use wherein the amino acid sequence of the LCVR is SEQ ID NO:9 and the amino acid sequence of the HCVR is SEQ ID NO:7. In an embodiment the present invention further provides the above use wherein the amino acid sequence of the light chain is SEQ ID NO:13 or SEQ ID NO:14. In an embodiment the present invention further provides the above use wherein the amino acid sequence of the heavy chain is SEQ ID NO:12. In an embodiment the present invention further provides the above use comprising two light chains, wherein the amino acid sequence of each light chain is SEQ ID NO:13, and two heavy chains, wherein the amino acid sequence of each heavy chain is SEQ ID NO:12. In an embodiment the present invention further provides the above use comprising two light chains wherein the amino acid sequence of each light chain is SEQ ID NO:14, and two heavy chains wherein the amino acid sequence of each heavy chain is SEQ ID NO:12. In an embodiment the present invention further provides the above use wherein the chronic pain is chronic osteoarthritis pain. In an embodiment the present invention further provides the above use wherein the chronic pain is chronic diabetic peripheral neuropathy pain. In an embodiment the present invention further provides the above use wherein the chronic pain is chronic low back pain. In an embodiment the present invention further provides the above use wherein the dose of antibody is a 750 mg starting dose, followed by a 500 mg dose every 2 weeks, for as long as the patient needs treatment for pain. In an embodiment the present invention further provides the above use wherein the chronic pain is refractory to two or more prior monotherapy and/or dual therapy treatment regimens. In an embodiment the invention provides for the use of an antibody according to the embodiments above for the manufacture of a medicament for the treatment of chronic pain. In an embodiment the invention provides for the use of an antibody according to the embodiments above for the manufacture of a medicament for the treatment of chronic pain, wherein the chronic pain is selected from chronic osteoarthritis pain, chronic diabetic neuropathy pain, and chronic low back pain.

### BRIEF DESCRIPTION OF FIGURES

FIG. 1. Pain efficacy of Antibody III in the rat meniscal tear model. Data are presented as mean±SEM where group size is n=6. Statistical comparisons: ANOVA and Dunnett's test for comparison to control IgG1 (*p<0.001) and Tukey's for comparison between 1 and 10 mg/kg Antibody III (*p<0.05). Abbreviations: ANOVA=analysis of variance; IgG1=immunoglobulin G1; SEM=standard error of the mean.

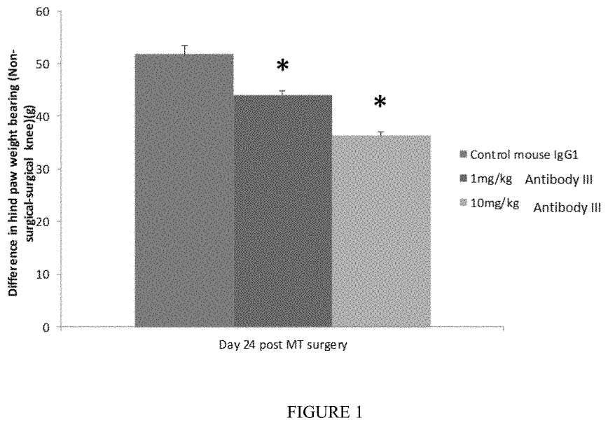

FIG. 2. Overview of 26-week, Phase 2, randomized, double-blind, placebo-controlled study that will compare Antibody I versus placebo.

As used herein chronic pain refers to pain which persists more than a day, or pain that recurs several times in a month. Chronic osteoarthritis pain, chronic diabetic neuropathy pain, and chronic low back pain and the identification of patients suffering from these conditions can be determined by methods known to the skilled artisan using established criteria, including those described herein. As used herein a patient is a human who has been diagnosed as having a condition or disorder in need of treatment with an antibody described herein. In those instances where the disorders which can be treated by the methods of the present invention are known by established and accepted classifications, such as osteoarthritis pain, diabetic neuropathic pain, or low back pain, their classifications can be found in various sources, and the International Classification of Diseases, Tenth Revision (ICD-10), provides classifications for the disorders described herein. The skilled artisan will recognize that there are alternative nomenclatures, nosologies, and classification systems for disorders described herein, including those as described in the DSM-IV and ICD-10, and that terminology and classification systems evolve with medical scientific progress.

As used herein, osteoarthritis pain expressly includes non-radicular (non-neuropathic pain). As used herein, neuropathic pain expressly includes radicular pain CLBP, DNP, and LSR. In embodiments, the pain is chronic pain, such as for example, chronic pain of both musculoskeletal as well as neuropathic origin that are treated by the present methods. In other embodiments, the pain treated by the present methods is visceral pain (such as, for example, chronic prostatitis, interstitial cystitis (bladder pain) or chronic pelvic pain).

Other embodiments provide a method of treating pain of nociceptive/inflammatory, neuropathic, nociplastic, or mixed etiologies. In other embodiments, the pain is chronic pain that is musculoskeletal or neuropathic in origin. Other types of pain treated by the present methods include post-surgical pain, rheumatoid arthritis pain, neuropathic pain, and osteoarthritis pain.

Exemplary types of pain include neuropathic pain, e.g., painful diabetic neuropathy, chemotherapy-induced peripheral neuropathy, lower back pain, trigeminal neuralgia, postherpetic neuralgia, sciatica, and complex regional pain syndrome; inflammatory pain, e.g., from rheumatoid arthritis, osteoarthritis, emporomandibular disorder; PDN or CIPN; visceral pain, e.g., from pancreatitis, inflammatory bowel disease, colitis, Crohn's disease, endometriosis, pelvic pain, and angina; pain selected from the group: cancer pain, burn pain, oral pain, crush and injury-induced pain, incisional pain, bone pain, sickle cell disease pain, fibromyalgia and musculoskeletal pain; or pain from hyperalgesia or allodynia.

Pain, as defined herein, expressly includes chronic pain of both musculoskeletal as well as neuropathic origin. “Post-surgical pain” (interchangeably termed “post-incisional” or “post-traumatic pain”) refers to pain arising or resulting from an external trauma such as a cut, puncture, incision, tear, or wound into tissue of an individual (including that that arises from all surgical procedures, whether invasive or non-invasive). As used herein, post-surgical pain does not include pain that occurs (arises or originates) without an external physical trauma. In some embodiments, post-surgical pain is internal or external (including peripheral) pain, and the wound, cut, trauma, tear or incision may occur accidentally (as with a traumatic wound) or deliberately (as with a surgical incision). As used herein, “pain” includes nociception and the sensation of pain, and pain can be assessed objectively and subjectively, using pain scores and other methods well-known in the art. Post-surgical pain, as used herein, includes allodynia (i.e., increased response to a normally non-noxious stimulus) and hyperalgesia (i.e., increased response to a normally noxious or unpleasant stimulus), which can in turn, be thermal or mechanical (tactile) in nature. In some embodiments, the pain is characterized by thermal sensitivity, mechanical sensitivity and/or resting pain. In some embodiments, the post-surgical pain comprises mechanically-induced pain or resting pain. In other embodiments, the post-surgical pain comprises resting pain. The pain can be primary or secondary pain, as is well-known in the art.

As used interchangeably herein, the term “patient,” “subject,” and “individual,” refers to a human. In certain embodiments, the patient is further characterized with a disease, disorder, or condition (e.g., pain) that would benefit from inhibition of TGF-alpha and epiregulin.

The term “treating” (or “treat” or “treatment”) means slowing, stopping, reducing, or reversing the progression or severity of a symptom, disorder, condition, or disease.

An effective amount can be determined by one skilled in the art by the use of known techniques and by observing results obtained under analogous circumstances. In determining the effective amount for a patient, a number of factors are considered, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The term “therapeutically effective amount” refers to the amount or dose of an antibody of this invention which, upon single or multiple dose administration to a patient, provides the desired treatment. An effective amount, in some embodiments, provides a clinically significant reduction in pain. A weekly, every two week, monthly, or quarterly parenteral (including, but not limited to, subcutaneous, intramuscular, and/or intravenous) dose can be from about 0.5 mg/kg to about 50 mg/kg.

A weekly, every two week, monthly, or quarterly parenteral (including, but not limited to, subcutaneous, intramuscular, and/or intravenous) dose can be from about 0.5 mg/kg to about 10 mg/kg, from about 1 mg/kg to about 10 mg/kg, from about 2 mg/kg to about 10 mg/kg, from about 3 mg/kg to about 10 mg/kg, from about 4 mg/kg to about 10 mg/kg, from about 5 mg/kg to about 10 mg/kg, from about 6 mg/kg to about 10 mg/kg, from about 7 mg/kg to about 10 mg/kg from about 8 mg/kg to about 10 mg/kg, from about 1 mg/kg to about 8 mg/kg, from about 2 mg/kg to about 8 mg/kg, from about 3 mg/kg to about 8 mg/kg, from about 4 mg/kg to about 8 mg/kg, from about 5 mg/kg to about 8 mg/kg, from about 6 mg/kg to about 8 mg/kg, from about 1 mg/kg to about 6 mg/kg, from about 2 mg/kg to about 6 mg/kg, from about 3 mg/kg to about 6 mg/kg, from about 4 mg/kg to about 6 mg/kg, from about 5 mg/kg to about 6 mg/kg, from about 1 mg/kg to about 5 mg/kg, from about 2 mg/kg to about 5 mg/kg, from about 3 mg/kg to about 5 mg/kg, from about 4 mg/kg to about 5 mg/kg, from about 1 mg/kg to about 4 mg/kg, from about 2 mg/kg to about 4 mg/kg, from about 3 mg/kg to about 4 mg/kg, from about 3.5 mg/kg to about 5 mg/kg, or about 4 mg/kg to about 5 mg/kg.

A weekly, every two weeks, monthly, or quarterly parenteral (including, but not limited to, subcutaneous, intramuscular, and/or intravenous) dose can be, for example, from about 50 mg to about 500 mg, from about 75 mg to about 500 mg, from about 100 mg to about 500 mg, from about 125 mg to about 500 mg, from about 250 mg to about 500 mg, from about 300 mg to about 500 mg, from about 350 mg to about 500 mg, from about 400 mg to about 500 mg, from about 450 mg to about 500 mg, from about 50 mg to about 400 mg, from about 75 mg to about 400 mg, from about 100 mg to about 400 mg, from about 125 mg to about 400 mg, from about 250 mg to about 400 mg, from about 300 mg to about 400 mg, from about 350 mg to about 400 mg, from about 50 mg to about 300 mg, from about 75 mg to about 300 mg, from about 100 mg to about 300 mg, from about 125 mg to about 300 mg, from about 150 mg to about 300 mg, from about 175 mg to about 300 mg, from about 200 mg to about 300 mg, from about 250 mg to about 300 mg, from about 50 mg to about 250 mg, from about 75 mg to about 250 mg, from about 100 mg to about 250 mg, from about 125 mg to about 250 mg, from about 150 mg to about 250 mg, from about 175 mg to about 250 mg, from about 200 mg to about 250 mg, from about 75 mg to about 250 mg, from about 50 mg to about 200 mg, from about 75 mg to about 200 mg, from about 100 mg to about 200 mg, from about 125 mg to about 200 mg, from about 150 mg to about 200 mg, from about 175 mg to about 200 mg, from about 50 mg to about 175 mg, from about 75 mg to about 175 mg, from about 100 mg to about 175 mg, from about 125 mg to about 175 mg, or from about 150 mg to about 175 mg.

However, doses below or above the doses mentioned herein are also envisioned, especially considering dosage considerations known to those skilled in the art and/or described herein. Progress of the patient being treated may be monitored by periodic assessment, and the dose adjusted accordingly if necessary.

The antibodies used in the methods of the present invention bind TGF-alpha and Epiregulin, and comprise a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

Furthermore, the antibodies used in the methods of the present invention bind TGF-alpha and Epiregulin, and comprise a light chain and a heavy chain, wherein the light chain comprises a light chain variable region (LCVR) and the heavy chain comprises a heavy chain variable region (HCVR), wherein the amino acid sequence of the LCVR is SEQ ID NO: 9 or SEQ ID NO: 10.

The antibodies used in the methods of the present invention bind TGF-alpha and Epiregulin, and comprise a light chain and a heavy chain, wherein the light chain comprises a light chain variable region (LCVR) and the heavy chain comprises a heavy chain variable region (HCVR), wherein the amino acid sequence of the HCVR is SEQ ID NO: 7.

The antibodies used in the methods of the present invention bind TGF-alpha and Epiregulin, and comprise a light chain and a heavy chain, wherein the light chain comprises a light chain variable region (LCVR) and the heavy chain comprises a heavy chain variable region (HCVR), wherein an amino acid sequence of the LCVR and an amino acid sequence of the HCVR is selected from the group consisting of:
(7) the LCVR is SEQ ID NO: 9 and the HCVR is SEQ ID NO: 7; and
(ii) the LCVR is SEQ ID NO: 10 and the HCVR is SEQ ID NO: 7.

The antibodies used in the methods of the present invention bind TGF-alpha and Epiregulin, and comprise a light chain and a heavy chain, wherein the light chain comprises a light chain variable region (LCVR) and the heavy chain comprises a heavy chain variable region (HCVR), wherein the amino acid sequence of the LCVR is SEQ ID NO: 9 and the amino acid sequence of the HCVR is SEQ ID NO: 7.

The antibodies used in the methods of the present invention bind TGF-alpha and Epiregulin, and comprise a light chain and a heavy chain, wherein the light chain comprises a light chain variable region (LCVR) and the heavy chain comprises a heavy chain variable region (HCVR), wherein the amino acid sequence of the LCVR is SEQ ID NO: 10 and the amino acid sequence of the HCVR is SEQ ID NO: 7.

The antibodies used in the methods of the present invention bind TGF-alpha and Epiregulin, and comprise a light chain and a heavy chain, wherein the amino acid sequence of the light chain is SEQ ID NO: 13 or SEQ ID NO: 14.

The antibodies used in the methods of the present invention bind TGF-alpha and Epiregulin, and comprise a light chain and a heavy chain, wherein the amino acid sequence of the heavy chain is SEQ ID NO: 12.

The antibodies used in the methods of the present invention bind TGF-alpha and Epiregulin, and comprise a light chain and a heavy chain, wherein an amino acid sequence of the heavy chain and an amino acid sequence of the light chain is selected from the group consisting of:
(i) the heavy chain is SEQ ID NO: 12 and the light chain is SEQ ID NO: 13, and
(ii) the heavy chain is SEQ ID NO: 12 and the light chain is SEQ ID NO: 14.

The antibodies used in the methods of the present invention bind TGF-alpha and Epiregulin, and comprise two light chains wherein the amino acid sequence of each light chain is SEQ ID NO: 13, and two heavy chains wherein the amino acid sequence of each heavy chain is SEQ ID NO: 12.

The antibodies used in the methods of the present invention bind TGF-alpha and Epiregulin, and comprise two light chains wherein the amino acid sequence of each light chain is SEQ ID NO: 14, and two heavy chains wherein the amino acid sequence of each heavy chain is SEQ ID NO: 12.

The antibodies used in the methods of the present invention bind TGF-alpha and Epiregulin, and comprise pharmaceutical compositions comprising the antibody as described herein, and at least one pharmaceutically acceptable carrier, diluent, or excipient.

The antibodies used in the methods of the present invention may comprise a pharmaceutical composition comprising the antibody as described herein, together with at least one pharmaceutically acceptable carrier, diluent, or excipient, and optionally other therapeutic ingredients.

The present invention also provides a method of treating chronic pain in a patient comprising administering to the patient an antibody of the present invention, as described herein, in separate, simultaneous or sequential combination with a standard of care.

Furthermore, the present invention provides methods of using an antibody as described herein, for use in therapy, wherein the antibody is to be administered in simultaneous or sequential combination with a standard of care. Preferably, the present invention provides methods of using an antibody as described herein for use in the treatment of chronic pain, wherein the antibody is to be administered in simultaneous or sequential combination with a standard of care.

The general structure of an “antibody” is very well-known in the art. For an antibody of the IgG type, there are four amino acid chains (two “heavy” chains and two “light” chains) that are cross-linked via intra- and inter-chain disulfide bonds. When expressed in certain biological systems, antibodies having unmodified human Fc sequences are glycosylated in the Fc region. Antibodies may be glycosylated at other positions as well. The subunit structures and three-dimensional configurations of antibodies are well known in the art. Each heavy chain is comprised of an N-terminal heavy chain variable region (“HCVR”) and a heavy chain constant region (“HCCR”). The heavy chain constant region is comprised of three domains (CH1, CH2, and CH3) for IgG, IgD, and IgA; and 4 domains (CH1, CH2, CH3, and CH4) for IgM and IgE. Each light chain is comprised of a light chain variable region (“LCVR”) and a light chain constant region (“LCCR”).

The variable regions of each light/heavy chain pair form the antibody binding site. The HCVR and LCVR regions can be further subdivided into regions of hypervariability, termed complementarity determining regions (“CDRs”), interspersed with regions that are more conserved, termed framework regions (“FR”). Each HCVR and LCVR are composed of three CDRs and four FRs, arranged from amino-terminus to carboxy-terminus in the following order: FR1, CDR1, FR2, CDR2, FR3, CDR3, FR4. Herein, the 3 CDRs of the heavy chain are referred to as “CDRH1, CDRH2, and CDRH3” and the 3 CDRs of the light chain are referred to as “CDRL1, CDRL2 and CDRL3.” The CDRs contain most of the residues which form specific interactions with the antigen. The assignment of amino acids to each domain is in accordance with well-known conventions [e.g., Kabat, “Sequences of Proteins of Immunological Interest,” National Institutes of Health, Bethesda, Md. (1991)].

An antibody used in the present invention may have a heavy chain constant region selected from any of the immunoglobulin classes (IgA, IgD, IgG, IgM, and IgE). Furthermore, an antibody used in the present invention contains an Fc portion which is derived from human IgG4 Fc region because of its reduced ability to bind complement factors as compared to other IgG sub-types.

An antibody may be derived from a single copy or clone, including e.g., any eukaryotic, prokaryotic, or phage clone. Preferably, an antibody used in the present invention exists in a homogeneous or substantially homogeneous population of antibody molecules. An full-length antibody comprises full length or substantially full length constant regions, including the Fc region. An “antigen-binding fragment” of such an antibody is any shortened form of a full length antibody that comprises the antigen-binding portion and retains antigen-binding capability. Such shortened forms include, e.g., a Fab fragment, Fab′ fragment or F(ab′) 2 fragment that includes the CDRs or the variable regions of the antibodies disclosed. Furthermore, such shortened antibody forms can be a single chain Fv fragment that may be produced by joining the DNA encoding the LCVR and HCVR with a linker sequence. (See, Pluckthun, The Pharmacology of Monoclonal Antibodies, vol. 113, Rosenburg and Moore eds., Springer-Verlag, New York, pp 269-315, 1994). The term “antibody” does not include such fragments unless otherwise indicated. An antibody used in the present invention can be produced using techniques well known in the art, e.g., recombinant technologies, phage display technologies, synthetic technologies or combinations of such technologies or other technologies readily known in the art.

An antibody used in the present invention is an engineered antibody that has been designed to have frameworks, hinge regions, and constant regions of human origin that are identical with or substantially identical (substantially human) with frameworks and constant regions derived from human genomic sequences. Fully human frameworks, hinge regions, and constant regions are those human germline sequences as well as sequences with naturally-occurring somatic mutations and those with engineered mutations. An antibody used in the present invention may comprise framework, hinge, or constant regions derived from a fully human framework, hinge, or constant region containing one or more amino acid substitutions, deletions, or additions therein. Further, an antibody used in the present invention is substantially non-immunogenic in humans.

A variety of different human framework sequences may be used singly or in combination as a basis for an antibody used in the present invention. Preferably, the framework regions of an antibody of the present invention are of human origin or substantially human (at least 95%, 97% or 99% of human origin.) The sequences of framework regions of human origin may be obtained from The Immunoglobulin Factsbook, by Marie-Paule Lafranc, Gerard Lefranc, Academic Press 2001, ISBN 012441351.

The framework sequence for an antibody used in the present invention serves as the “donor” variable framework region and can be used to create additional antibodies with the same CDRs specified herein using methodology known in the art. Furthermore, the framework sequence for an antibody used in the present invention can be compared to other known human framework sequences to generate additional antibodies. Thus, this information can be used to “back-mutate” another selected homologous human framework region to the donor amino acid residue at these positions. Further, any “rare” amino acids can be detected in additional human frameworks such that the consensus or donor amino acid residue can be used at the relevant position.

“TGF-alpha” or “human TGF-alpha” refers to human TGF-alpha protein (SEQ ID NO: 18).

“Epiregulin” or “human Epiregulin” refers to human Epiregulin protein (SEQ ID NO: 33). Met-human Epiregulin (SEQ ID NO: 22) is used in in vitro experiments herein. References to the ability of the antibodies as described herein, to bind or to neutralize human Epiregulin pertain also to their ability to bind and to neutralize human met-Epiregulin in in vitro experiments.

The following examples may be performed essentially as described below.

### EXAMPLES

### Example 1: Production of Antibodies

Antibodies I and II can be made and purified as follows. An appropriate host cell, such as HEK 293 or CHO, is either transiently or stably transfected with an expression system for secreting antibodies using an optimal predetermined HC:LC vector ratio or a single vector system encoding both HC, such as SEQ ID NO: 15, and LC, such as SEQ ID NO: 16 or SEQ ID NO: 17. Clarified media, into which the antibody has been secreted, is purified using any of many commonly-used techniques. For example, the medium may be conveniently applied to a Protein A or G column that has been equilibrated with a compatible buffer, such as phosphate buffered saline (pH 7.4). The column is washed to remove nonspecific binding components. The bound antibody is eluted, for example, by pH gradient (such as 0.1 M sodium phosphate buffer pH 6.8 to 0.1 M sodium citrate buffer pH 2.5). Antibody fractions are detected, such as by SDS-PAGE, and then are pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, or hydroxyapatite chromatography. The purity of the antibody after these chromatography steps is greater than 99%. The product may be immediately frozen at −70° C. or may be lyophilized. The amino acid sequences for these antibodies are provided below.

### SEQ ID NOs

| | Heavy | Light | | |
|---|---|---|---|---|
| Antibody | Chain | Chain | HCVR | LCVR |
| I | 12 | 13 | 7 | 9 |
| II | 12 | 14 | 7 | 10 |
| III | 31 | 32 | 8 | 11 |

| Antibody | HCDR1 | HCDR2 | HCDR3 | LCDR1 | LCDR2 | LCDR3 |
|---|---|---|---|---|---|---|
| I | 1 | 2 | 3 | 4 | 5 | 6 |
| II | 1 | 2 | 3 | 4 | 5 | 6 |
| III | 1 | 2 | 3 | 4 | 5 | 6 |

### Formulations:

The drug product of Antibody I is supplied for clinical trial use as a lyophilized powder in a glass vial. The vial contents are be reconstituted/diluted with Sterile 0.9% Sodium Chloride, United States Pharmacopeia (USP). The lyophilized drug product Antibody I is composed of Antibody I and the excipients sodium citrate, citric acid, polysorbate 80, and sucrose. The vial is manufactured to deliver 75 mg of Antibody I. Reconstituting/diluting the vial contents with 3.2 mL of Sterile 0.9% Sodium Chloride, USP, produces a clear solution consisting of 25 mg/mL of Antibody I at a pH of 6.0.

### Example 2: Affinity Binding Measurement by Surface Plasmon Resonance (BIAcore) for Antibody I

Biacore T2000 instrument (BIAcore® AB, Upsala, Sweden), reagents and Biacore T2000 Evaluation Software Ver 4.1 are used for the Surface Plasmon Resonance analysis. A CMS chip is prepared using manufacturer's EDC/NHS amine coupling method. The surfaces of all four flow cells are activated by injecting a 1:1 mixture of EDC/NHS for 7 minutes at 10 μL/min. Goat anti-human Fc y specific antibody is diluted to 50 μg/ml in 10 mM acetate, pH 4.0 buffer and immobilized for approximately 10000 RU onto all four flow cells by 7 minute injection at a flow rate of 10 μL/min. Un-reacted sites are blocked with a 7 minute injection of ethanolamine at 10 μL/min. Injections of 3×20 seconds of glycine pH 1.5 at 30 μL/min are used to remove non-covalently associated protein. The running buffer is HBS-EP [10 mM HEPES, 150 mM Sodium Chloride, 3 mM EDTA, 0.005% Polysorbate 20].

In study 1, Antibody I is diluted to 50 μg/mL in running buffer, and approximately 400-600 RU is captured in flowcell 2. Human TGF-alpha (SEQ ID NO: 18), rat TGF-alpha (SEQ ID NO: 20), met-human Epiregulin (SEQ ID NO: 22), and cynomolgus Epiregulin (SEQ ID NO: 24) are diluted from 100 μg/mL to 200 nM in running buffer and then two-fold serially diluted in running buffer to 6.25 nM. Mouse Epiregulin (SEQ ID NO: 23) is diluted from 100 μg/mL to 4 μM in running buffer and then two-fold serially diluted in running buffer to 125 nM. Duplicate injections of each ligand concentration are injected at 30 μL/min for 300 seconds followed by a dissociation phase. The dissociation phase is 1800 seconds for human and rat TGF-alpha, 1200 seconds for human and cynomolgus Epiregulin, and 120 seconds for mouse Epiregulin. Regeneration is performed by injecting 10 mM glycine pH 1.5 for 3×20 seconds at 30 μL/min over all flowcell.

In study 2, Antibody III is diluted to 100 μg/mL in running buffer, and approximately 400-600 RU is captured in flowcell 2. Mouse TGF-alpha (SEQ ID NO: 19), is diluted from 100 μg/mL to 200 nM in running buffer and then two-fold serially diluted in running buffer to 6.25 nM. Mouse Epiregulin (SEQ ID NO: 23) is diluted from 100 μg/mL to 4 μM in running buffer and then two-fold serially diluted in running buffer to 125 nM. Duplicate injections of each ligand concentration are injected at 30 μL/min for 300 seconds followed by a dissociation phase. The dissociation phase is 1800 seconds for mouse TGF-alpha, and 120 seconds for mouse Epiregulin. Regeneration is performed by injecting 10 mM glycine pH 1.5 for 30 seconds at 30 μL/min over all flowcell.

Reference-subtracted data are collected as Fc2-Fc1. The measurements are obtained at 25° C. The on-rate (kₒₙ) and off-rate (k_{off}) for each ligand are evaluated using a “1:1 (Langmuir) Binding” binding model. The affinity (K_{D}) is calculated from the binding kinetics according to the relationship: K_{D}=k_{off}/kₒₙ.

TABLE 1
Binding Parameters for Antibody I

| | | On Rate (kₒₙ) | Off Rate (k_{off}) | Affinity | |
|---|---|---|---|---|---|
| | | (M⁻¹s⁻¹) | (s⁻¹) | (K_{D}^{a}) | |
| Ligand | Species | (±SD) | (±SD) | (±SD) | |
| TGF-alpha | Human | 4.18 ± 0.28 × 10⁵ | 4.09 ± 0.96 × 10⁻⁵ | 97.6 ± 20.6 | pM |
| | Rat | 3.78 ± 0.39 × 10⁵ | 2.66 ± 0.74 × 10⁻⁵ | 70.5 ± 19.4 | pM |
| Epiregulin | Human | 4.91 ± 0.42 × 10⁵ | 6.31 ± 0.55 × 10⁻⁴ | 1.29 ± 0.03 | nM |
| | Cynomolgus | 6.73 ± 0.71 × 10⁵ | 7.05 ± 0.23 × 10⁻⁴ | 1.05 ± 0.09 | nM |
| | Mouse | 4.10 ± 1.15 × 10⁴ | 1.33 ± 0.16 × 10⁻² | 342 ± 136 | nM |

^{a}Calculated as K_{D} = k_{off}/kₒₙ

TABLE 2
Binding Parameters for Antibody III

| | On Rate (kₒₙ) | Off Rate (k_{off}) | Affinity | |
|---|---|---|---|---|
| | (M⁻¹s⁻¹) | (s⁻¹) | (K_{D}^{a}) | |
| Ligand | (±SD) | (±SD) | (±SD) | |
| Mouse | 5.41 ± 0.50 × 10⁵ | 2.02 ± 0.54 × 10⁻⁵ | 38.0 ± 13.6 | pM |
| TGF-alpha | | | | |
| Mouse | 6.55 ± 0.38 × 10⁴ | 1.41 ± 0.09 × 10⁻² | 215 ± 15 | nM |
| Epiregulin | | | | |

^{a}Calculated as K_{D} = k_{off}/kₒₙ

Thus, Antibody I specifically binds TGFα and epiregulin and binds very weakly to epigen. The apparent binding kinetics and affinity of Antibody I to TGFα, epiregulin, and epigen from various species were measured by surface plasmon resonance (SPR). Transforming growth factor α, epiregulin, and epigen from various species produced a concentration-dependent binding response with Antibody I that had a strong affinity for human and rat TGFα (K_{d} of 97.6±20.6 pM and 70.5±19.4 pM at 25° C., respectively). The binding kinetics of cynomolgus monkey and mouse TGFα were not measured separately, as they are 100% identical to human and rat TGFα, respectively. The binding kinetics of rabbit TGFα were not measured separately, as the Antibody I epitope is identical in rabbit and human TGFα. Antibody I showed strong affinity for human and cynomolgus monkey epiregulin and affinity for mouse epiregulin (K_{d} of 1.29±0.03 nM, 1.05±0.09 nM, and 342±136 nM at 25° C., respectively). The binding kinetics of rat epiregulin were not measured separately, as the Antibody I epitope is identical in rat and mouse epiregulin. Antibody I showed very weak affinity for human and mouse epigen (K_{d}>2 μM and 493±205 nM at 25° C., respectively).

Since Antibody I is a humanized monoclonal antibody, its use for evaluation in animal models of pharmacology is limited due to the likelihood of generating an immune response to the compound by the animals. Hence, a mouse monoclonal antibody, Antibody III, was used to determine the effect of neutralizing TGFα and epiregulin in an animal model of OA pain. The binding kinetics of Antibody III to mouse TGFα, epiregulin, and epigen were also measured by SPR. Antibody III showed strong affinity for TGFα and weak affinity for epiregulin and epigen, with associated K_{d} values of 38.0±13.6 pM, 215±15 nM, and 365±39 nM at 25° C., respectively.

### Example 3: Internalization of EGF Target Ligands in the Human Colon Carcinoma Cell Line HT-29

Conjugation of Alexa Fluor® 488 to antibodies

Alexa Fluor® 488 is conjugated to Antibody I and Control IgG according to the manufacturer's protocol. Protein is diluted to 2 mg/mL in PBS. To 0.5 mL of this 2 mg/mL solution, 50 μL of 1M sodium bicarbonate pH 9 is added. The protein solution is then transferred to a vial of dye and stirred at room temperature for 1 hour. The labeled protein is purified using the Bio-Rad BioGel P-30 resin included with the labeling kit.

### In Vitro Internalization Assay

In study 1, 10,000 HT-29 cells, a colon adenocarcinoma cell line known to express TGF-alpha and Epiregulin, are seeded per well of a 96 well plate and allowed to incubate overnight in complete media [Dulbecco's Modified Eagle's Medium/F12 (Ham) Medium (1:1) (“DMEM/F12”) containing L-glutamine, 10% heat-inactivated fetal bovine serum (“FBS”), lx antibiotic, and 2.438 g/L sodium bicarbonate]. The next day, the cells are washed with PBS containing 0.1% BSA and then incubated with an Alexa Fluor® 488 conjugated Antibody I or Control IgG in PBS with 0.1% BSA at concentrations ranging from 0 to 88 ug/mL for 2 hours at 37° C. in a tissue culture incubator. Following the incubation period, the cells are washed in PBS with 0.1% BSA several times and then fixed with 4% formaldehyde for analysis. The quantitation of internalization is done as follows: 500 cells/well are collected with a Cellomics Arrayscan VTI (Thermo Scientific). Image analysis is performed with “Compartment al analysis” Bioapplications of the system. Cell nuclei are identified with a Hoechst stain (blue). Two regions of interest (ROI) are set to collect fluorescent signals from intracellular spots (red) and total green fluorescence (both red and blue) obtained from the masked image. The number, area and fluorescent intensity from each spot and cell are calculated. The mean spot total intensity of intracellular spots (red) is chosen for measuring Antibody I induced internalization.

In study 2, 10,000 HT-29 cells are prepared as previously described, and Alexa Fluor® 488 conjugated Antibody I or Control IgG in PBS containing 0.1% BSA is added to the cells at 40 ug/mL. Cells are incubated at 37° C. in a tissue culture incubator for various times ranging from 0-120 minutes, then washed with PBS containing 0.1% BSA several times and fixed with 4% formaldehyde for analysis. The quantification of signal is performed essentially as previously described.

TABLE 3a
Study 1 - Mean Ringspot Total Intensity of Fluorescence

| Dose (ug/ml) | 88 | 44 | 22 | 11 | 5.5 |
|---|---|---|---|---|---|
| Control IgG | 2440 ± 199 | 1808 ± 207 | 1763 ± 68 | 1391 ± 76 | 1357 ± 63 |
| Antibody I | 24809 ± 4343 | 17451 ± 217 | 15135 ± 131 | 11516 ± 54 | 8474 ± 269 |

Mean ± SEM

TABLE 3b
Study 1 - Mean Ringspot Total Intensity of Fluorescence

| Dose (ug/ml) | 2.75 | 1.38 | 0.69 | 0.34 | 0 |
|---|---|---|---|---|---|
| Control IgG | 1570 ± 70 | 1473 ± 7 | 1483 ± 90 | 1407 ± 41 | 1630 ± 155 |
| Antibody I | 6503 ± 262 | 4349 ± 186 | 3440 ± 96 | 2432 ± 62 | 1460 ± 84 |

Mean ± SEM

The results from the imaging analysis of study 1 determined that the fluorescence signal was internalized into the cell and was dose dependent with Antibody I, but not with the Control IgG (Table 3a and Table 3b).

TABLE 4
Study 2 - Mean Ringspot Total Intensity of Fluorescence

| | Time post addition (min) | | | | | |
|---|---|---|---|---|---|---|
| | 120 | 60 | 30 | 15 | 5 | 0 |
| Control IgG | 177 ± 29 | 167 ± 23 | 124 ± 10 | 126 ± 18 | 116 ± 4 | 94 ± 11 |
| Antibody I | 4449 ± 866 | 4131 ± 1688 | 1494 ± 66 | 717 ± 72 | 261 ± 17 | 89 ± 1 |

Mean ± SEM

The results from study 2 demonstrated that Antibody I was internalized rapidly and the internalization was complete by 2 hours post addition to cells (Table 4). Antibody I induced internalization of target on HT-29 cells in vitro in a time dependent manner (Table 4). The results above indicate that Antibody I binds to the membrane-bound ligands and promotes their internalization in a dose- and time-dependent manner, with complete internalization of Antibody I (and presumably ligand) within 2 hours of incubation at 37° C.

### Example 4: Measurement of Neutralization of EGFR Ligand Stimulated Cell Proliferation in a Myofibroblast Cell Line

A clonal mouse myofibroblast cell line (“MFc7”) is used to test the ability of the antibodies of the present invention to block the proliferative activity of EGFR ligands. The seven ligands that can activate the EGFR are TGF-alpha (TGFA), Epiregulin (EREG), EGF, Heparin-Binding EGF (HB-EGF), Epigen (EPGN), Amphiregulin (AREG) and Betacellulin (BTC). The EGFR ligands share a structural motif, the EGF-like domain, characterized by three intramolecular disulfide bonds that are formed by six similarly spaced conserved cysteine residues. Proliferative activity is determined by Bromodeoxyuridine (“BrDU”) incorporation and is measured with a colorimetric BrDU ELISA kit according to the manufacturer's instructions.

First, 2,000 MFc7 cells/well are plated in a tissue culture treated 96 well microplate in 0.1 mL of Dulbecco's Modified Eagle's Medium/F12 (Ham) Medium (1:1) (“DMEM/F12”) containing L-glutamine, 10% heat-inactivated FBS, lx antibiotic, and 2.438 g/L sodium bicarbonate. Cells are allowed to attach for 6 hours, and then the medium is removed and replaced with 0.1 mL of serum free DMEM/F12 containing 0.1% BSA for serum starvation overnight. The next day, serial dilutions of the EGFR ligands are made with serum free media containing 0.1% BSA in 96 well polypropylene plates in a volume of 0.12 mL/well from concentrations ranging from 0.001 to 3000 ng/mL. Following dilutions, medium is removed from serum starved cells and then stimulated with EGFR ligand for 24 hrs. Following stimulation, the cells are pulsed with BrDU for 4 hrs and then analyzed with a colorimetric BrDU ELISA kit according to the manufacturer's instructions.

In testing the specificity of Antibody I to EGFR ligands, serial dilutions of 2× or 3× of the antibody are made in 96 well polypropylene plates in a volume of 0.06 mL/well from concentrations ranging from 3000 nM to 0.059 nM. Following serial dilutions of the antibody, 0.06 mL of the EGFR ligand is added per well. The plate is then incubated at 37° C. in a humidified tissue culture incubator for 30 minutes. Following incubation, 0.1 mL of the solution is transferred per well to the cells. The cells are stimulated for 24 hours. Following stimulation, the cells are pulsed with BrDU for 4 hours and then analyzed with a colorimetric BrDU ELISA kit. Absorbance values (450 nM-690 nM) are generated on a SpectraMax 190 plate reader (Molecular Devices) and data are analyzed.

TABLE 5
MFc7 Assay

| | EC50 Range | IC50 (nM) | IC50 (nM) |
|---|---|---|---|
| EGFR Ligand | (pM) | Antibody I | Antibody III |
| Human TGF-alpha^{a} | 11-12 | 0.46 ± 0.03 | 0.52 ± 0.04 |
| Human Epiregulin | 78-282 | 3.15 ± 1.04 | 1.12 ± 0.36 |
| Human Epigen | 3797-18987 | 807 ± 577 | nd^{b} |
| Human EGF | 0.3-2.4 | >2000 | nd^{b} |
| Human HBEGF | 30-39 | >2000 | nd^{b} |
| Human Betacellulin | 1.8-3.2 | >2000 | nd^{b} |
| Human Amphiregulin | 273-2727 | >2000 | nd^{b} |
| Rat TGF-alpha | 13-13.8 | 0.19 ± 0.06 | 0.13 ± 0.01 |
| Mouse Epiregulin | 163-320 | 334 ± 41 | 214 ± 49 |

^{a}Human EGFR ligands were at a concentration of 0.5 nM when tested with Antibody I, except for Amphiregulin (60 nM) and Epigen (100 nM)
Rat TGF-alpha and Mouse Epiregulin were used at 0.5 nM
^{b}nd, not determined

Mouse Epiregulin and rat TGF-alpha, as well as all of the human EGFR ligands except for Epigen and Amphiregulin were found to be potent stimulators of cell proliferation in the assay (Table 5). Antibody I and Antibody III have high affinity to human and rat TGF-alpha and human Epiregulin activity (Table 5).

Table 5 summarizes the calculated EC50 values for the EGFR ligands tested and the absolute IC50 values for the antibodies to those ligands. The calculated average IC50 for Antibody I was 0.46±0.03 nM to human TGF-alpha and 3.15±1.04 nM to human Epiregulin. The calculated IC50 average for Antibody III was 0.52±0.04 nM to human TGF-alpha and 1.12±0.36 nM to human Epiregulin. The calculated average IC50 value for Antibody III was 0.13±0.01 nM to rat TGF-alpha and 214±49 nM to mouse Epiregulin. Thus, Antibody I and Antibody III have high affinity and are selective with full neutralizing activity against human TGF-alpha and human Epiregulin.

In summary, Epidermal growth factor receptor ligands are potent mitogenic factors for cells of many lineages, including fibroblastic cells. To determine the neutralizing efficacy and specificity of Antibody I, its effect on proliferation of myofibroblast cells was evaluated in vitro. Individual recombinant ligands were used to stimulate proliferation of the myofibroblast cells and to determine their relative 50% effective concentration (EC₅₀) for such. Subsequently, submaximal concentrations of individual ligands and various concentrations of Antibody I were evaluated in the assay to quantitate the inhibitory activity for specific ligands. Antibody I has potent neutralizing activity toward human, mouse, rat, and cynomolgus monkey TGFα (50% inhibitory concentration [IC₅₀] values <0.5 nM) and toward human and cynomolgus monkey epiregulin (IC₅₀ values <3.5 nM). Antibody I has weaker neutralizing activity toward mouse and rat epiregulin (IC₅₀ values of <350 nM) and human, cynomolgus monkey, and rat epigen (IC₅₀ values <850 nM) and no measurable activity toward the other EGFR ligands tested (IC₅₀ values >2000 nM).

In a similar way, neutralizing activity of the mouse antibody, Antibody III, was evaluated in the myofibroblast proliferation assay. Antibody III also potently inhibited human and rat TGFα and human epiregulin (IC₅₀ values of 0.521±0.037 nM, 0.131±0.012 nM, and 1.12±0.36 nM, respectively) and activity to mouse epiregulin (IC₅₀ value of 214±49 nM). There was no measurable activity toward the other mouse EGFR ligands tested.

Thus, Antibody I is a high-affinity humanized immunoglobulin G4 (IgG4) monoclonal antibody that binds to key residues in the C-terminal regions of human TGFα and epiregulin, preventing their binding to and activation of EGFR. Engagement of Antibody I results in internalization of the membrane-bound proforms of both TGFα and epiregulin and neutralization of the mature (soluble) ligand activity. Clinical data show that Antibody I was well tolerated in healthy subjects after a single dose of up to 750 mg and had an acceptable tolerability profile in patients with moderate to severe diabetic nephropathy (DN) after multiple doses up to 750 mg intravenously (IV) every 3 weeks over a period of 3 months. The safety profile of Antibody I is differentiated from that of EGFR antibodies or EGFR tyrosine kinase inhibitors, with low incidence of skin and GI adverse events (Aes). Dose/concentration-dependent increase in total epiregulin concentration after Antibody I infusion suggests that it binds to epiregulin in vivo. The molecular, pharmacological, and clinical properties of Antibody I support the concept that this antibody possesses the activity, availability, safety and tolerability to advantageously treat diseases and disorders responsive to inhibition of both TGFα and epiregulin.

The present disclosure provides that Antibody I is useful for the treatment of chronic pain, including nociceptive, neuropathic, and mixed pain, and in particular treatment of osteoarthritis (OA), or diabetic peripheral neuropathy (DPNP), or chronic low back pain. Furthermore, the present disclosure provides that Antibody I is useful for the treatment of osteoarthritis (OA), or diabetic peripheral neuropathy (DPNP), or chronic low back pain refractory to two or more prior monotherapy and/or dual therapy treatment regimens. Antibody III, which shares the same CDRs as Antibody I, was tested in a preclinical model of Osteoarthritis pain, and discovered to have efficacy in the treatment of pain in this model as described below. This supports the concept that Antibody I is useful for the treatment of chronic pain disorders as described herein.

### Example 5: The In Vivo Effect of Anti-TGF Alpha Antibody III in the Meniscal Tear Model of Osteoarthritic Knee Pain in the Rat

OA is a chronic, debilitating, joint disease with resulting pain in the affected joint. In this study, Antibody III was tested for its ability to prevent disease progression and reduce osteoarthritic-like knee pain in the rat MT model of OA. MT is a well described model of OA where joint destruction and pain occur after surgical destabilization of the knee joint by transaction of the medial collateral ligament and medial meniscus. OA disease progression was assessed by histology. Pain was measured as a difference in weight bearing between the surgical knee with induced OA and the unoperated contralateral knee of the same animal. Antibody III treatment at 1 and 10 mg/kg showed no effect on OA disease progression, but showed statistically significant pain reduction in a dose-dependent manner compared to a control IgG1 antibody.

For this study, 48 male Lewis rats (Harlan, Indianapolis, Ind.) aged 28 to 29 weeks and weighing 345 to 430 grams were used. The rats were housed in groups of 3 per cage and maintained in a constant temperature with a 12-hour light/12-hour dark cycle. Animals had free access to food and water at all times except during data collection. Rats were randomized into 3 groups of 16 by body weight, and OA was induced in the rats by surgical sectioning of the medial collateral ligament and meniscus of the right knee joint. No surgical sectioning was done on the left knee joint. Rats were given SC doses of either 10 mg/kg control mouse IgG1 antibody or 1 or 10 mg/kg Antibody III. Dosing started on the day of surgery, just prior to surgery, and rats were dosed once a week until study end. Dose volume was 2 mL/kg and dosing continued once per week via subcutaneous injection until study end 4 weeks post-surgery. The last doses were given 1 week prior to necropsy, which occurred 4 weeks after surgery. At necropsy, right and left knee joints were harvested, fixed in 10% zinc formalin, and then sent to BolderBioPATH for embedding, sectioning, histological staining, and scoring by a board-certified veterinary pathologist. To assess pain in this model, 6 rats were randomly selected from each treatment group and assessed for pain on Day 24 of the study using incapacitance testing (static differential weight bearing). This measured differences in hind paw weight bearing between the surgical and unoperated knees. For this study, reported values were the average of 3 separate measurements, each measured over 1 second for each rat. The operator was blinded to the treatment groups of the selected rats.

Pain data are presented as means with standard error of the mean (±SEM). Data were evaluated by one-way analysis of means. Groups were compared using Dunnett's test while the Tukey-Kramer HSD test was utilized for pairwise comparison with the JMP statistical analysis program (SAS Institute Inc., NC). Differences were considered significant if the p value was less than 0.05 (p<0.05).

For histology data, BolderBioPATH performed the analysis. Data was analyzed using the Student's t-test or Mann-Whitney U test (non-parametric). If appropriate, data was further analyzed across all groups by a one-way analysis of variance (ANOVA) or Kruskal-Wallis test (non-parametric), along with the appropriate multiple comparison post-test. Differences were considered significant if the p value was less than 0.05 (p<0.05).

Histologic scoring of knee joints from this study indicated that treatment with Antibody III (1 or 10 mg/kg) did not significantly affect lesions of medial meniscal tear-induced OA in rats when compared to control IgG1. However, Antibody III significantly reduced pain compared to control IgG1 at both 1 and 10 mg/kg (p<0.001 by Dunnett's test; (Error! Reference source not found.). The 10-mg/kg dose of Antibody III was also significantly different from the 1-mg/kg dose (p<0.05, Tukey Kramer Honest Significant Difference test). The data provide surprising and unexpected evidence that Antibody III is effective at reducing pain, as measured by differences in weight bearing in the rat meniscal tear model.

### Effects in Humans

Clinical trial data represented in this section were generated in accordance with the principles of Good Clinical Practice (GCP). Two clinical trials have been completed, in which 93 subjects have been exposed to Antibody I to date. Forty-two healthy subjects received single doses (IV or SC) in Study I5V-MC-TGAA (TGAA). Fifty-one patients received multiple doses by IV infusion in Study I5V-MC-TGAB (TGAB). The designs of these studies are summarized in Table 6.

TABLE 6
Listing of Clinical Pharmacology Studies

| Brief Description | Trial | | Dosing | Safety |
|---|---|---|---|---|
| of Study | Alias | Population | Regimen | Assessments |
| Placebo- | TGAA | 56 healthy | 0.1, 1, 10, 50, 250, | Routine clinical |
| controlled, single- | | subjects | or 750 mg Antibody | safety |
| dose, dose- | | | I (n = 6 per dose) | assessments, |
| escalation study in | | | versus placebo (n = 2 | inspection of skin |
| healthy volunteers | | | per dose) | and eyes, |
| | | | IV single dose | pulmonary |
| | | | 50 mg SC single | diffusing |
| | | | dose (n = 6 Antibody | capacity, and |
| | | | I, n = 2 placebo) | ADA assessment |
| Phase ½ | TGAB | 60 patients | Part A: multiple | Routine clinical |
| randomized, | | with | ascending doses of | safety |
| double-blind, | | diabetic | 10, 100, and 750 mg | assessments, |
| placebo- | | nephropathy | Antibody I (n = 4 per | DLCO, Aes, |
| controlled, | | | dose) versus placebo | ADA assessment, |
| multiple IV | | | (n = 1 per dose) IV | and safety |
| infusion dose- | | | every 3 weeks for 2 | laboratory testing |
| escalation (Part A) | | | doses; | (including |
| and multiple IV | | | Part B: | chemistry, |
| infusion parallel- | | | 50 mg (n = 14), | hematology, and |
| dose (Part B) study | | | 250 mg (n = 13), or | urinalysis). |
| in patients with | | | 750 mg Antibody I | |
| diabetic | | | (n = 12) or placebo | |
| nephropathy | | | (n = 6) every 3 weeks | |
| | | | for up to 5 doses | |

Abbreviations: ADA = anti-drug antibody; AE = adverse event; DLCO = diffusion capacity measurements; IV = intravenous; n = number of subjects, SC = subcutaneous; Study TGAA = Study I5V-MC-TGAA; Study TGAB = Study I5V-MC-TGAB.

### Example 6: Clinical Pharmacology Pharmacokinetics

After single and multiple IV dosing, Antibody I exhibited nonlinear pharmacokinetics, indicative of target-mediated drug disposition (Tables 6.1, 6.2, and 6.3). At low doses, the apparent plasma clearance of Antibody I was significantly larger, with a relatively short T_{1/2}. As the dose of Antibody I increased, the T_{1/2} of Antibody I approached 432 hours (18 days), consistent with what is commonly observed for monoclonal antibodies that target membrane-bound proteins. In DN patients, Antibody I mean exposure (AUC from time zero to time t, where t is the last time point with a measurable concentration [AUC₀₋ₜₗₐₛₜ]) increased from 2250 μg*day/mL after a single dose (Table) to 4880 μg*day/mL after the fifth dose (Table 6.) for 750-mg IV infusions, resulting in an approximately 2-fold accumulation when administered every 3 weeks. After a single IV infusion of 750 mg, the maximum concentration (Cₘₐₓ) and exposure (AUC₀₋ₜₗₐₛₜ) were comparable in healthy subjects and DN patients (mean Cₘₐₓ of 265 μg/mL and 368 μg/mL; mean AUC₀₋ₜₗₐₛₜ of 3040 μg·day/mL and 2250 μg·day/mL in healthy subjects and DN patients, respectively). Population PK modeling showed that the pharmacokinetics of Antibody I were similar in healthy subjects and DN patients. The calculated bioavailability of Antibody I after SC dose administration was 38%; however, the evaluated dose (50 mg) lies in the range of doses where pharmacokinetics are nonlinear. Therefore, bioavailability in the clinically relevant linear PK range is likely to be higher.

TABLE 6.1
Summary of Noncompartmental Antibody I Pharmacokinetic Parameters after
Single IV and SC Doses of Antibody I in Healthy Volunteers in Study TGAA

| | Geometric Mean (CV % Geometric Mean) | | | | | | | | | | | | | |
|---|---|---|---|---|---|---|---|---|---|---|---|---|---|---|
| | Antibody I | | Antibody I | | Antibody I | | Antibody I | | Antibody I | | Antibody I | | Antibody I | |
| | 0.1 mg IV | | 1 mg IV | | 10 mg IV | | 50 mg IV | | 250 mg IV | | 750 mg IV | | 50 mg SC | |
| N | 6 | | 6 | | 6 | | 6 | | 6 | | 6 | | 6 | |
| Cₘₐₓ | 0.036 | (12) | 0.390 | (24) | 3.76 | (18) | 18.4 | (10) | 105 | (19) | 265 | (23) | 3.42 | (54) |
| (μg/mL) | | | | | | | | | | | | | | |
| T_{1/2} | NC | (NA) | NC | (NA) | 1.63 | (11) | 3.76 | (23) | 8.31 | (52) | 18.0^{a} | (5.4) | 3.42^{a} | (19) |
| (days) | | | | | | | | | | | | | | |
| AUC₀₋ₜₗₐₛₜ | 0.0069 | (18) | 0.284 | (43) | 9.50 | (19) | 113 | (18) | 887 | (35) | 3040 | (23) | 39.2 | (55) |
| (μg*day/mL) | | | | | | | | | | | | | | |

| | | | | | | | | | | | | |
|---|---|---|---|---|---|---|---|---|---|---|---|---|
| AUC_{0-∞} | NC | NC | 9.67 | (19) | 114 | (18) | 911 | (38) | 3310 | (26) | 39.1 | (62) |
| (μg*day/mL) | | | | | | | | | | | | |
| CL | NC | NC | 1.03 | (19) | 0.439 | (18) | 0.274 | (38) | 0.227^{a} | (26) | 1.28^{a} | (62) |
| (L/day) | | | | | | | | | | | | |

| | | | | | | | | | | | |
|---|---|---|---|---|---|---|---|---|---|---|---|
| Vₛₛ | NC | NC | 2.64 | (11) | 2.92 | (13) | 3.52 | (18) | 5.48^{a} | (21) | NC |
| (L) | | | | | | | | | | | |

| F (%)^{b} | NC | NC | NC | NC | NC | NC | 38.1 | |
|---|---|---|---|---|---|---|---|---|
| Tₘₐₓ^{c} | — | — | — | — | — | — | 3.00 | (3.00-7.00) |
| (days) | | | | | | | | |

Abbreviations: AUC₀₋ₜₗₐₛₜ = area under the concentration-time curve from time zero to time t, where t is the last time point with a measurable concentration; AUC_{0-∞} = area under the concentration-time curve from time zero to infinity; CL = total body clearance of drug, which is the absolute clearance for IV administration and the apparent clearance (CL/F) for SC administration; Cₘₐₓ = maximum serum concentration; CV = coefficient of variation; F = bioavailability; IV = intravenous; N = number of subjects in dose group; NA = not applicable; NC = not calculable; SC = subcutaneous; Study TGAA = Study I5V-MC-TGAA; Tₘₐₓ = time to maximum concentration; T_{1/2} = elimination half-life; Vₛₛ = volume of distribution at steady state.
^{a}Data from 5 subjects included in these reported values.
^{b}F = (Antibody I 50 mg SC Mean AUC_{0-∞}/Antibody I 50 mg IV Mean AUC_{0-∞})*100.
^{c}Median (range).

### Source: I5V-MC-TGAA Table 14.2.1.2. Summary of Derived Antibody I Pharmacokinetic Parameters.

TABLE 6.2
Summary of Noncompartmental Antibody I Pharmacokinetic
Parameters after Single IV Doses of Antibody I in
Diabetic Nephropathy Patients in Study TGAB (Part A)

| | Antibody I 10 mg IV | | Antibody I 100 mg IV | | Antibody I 750 mg IV | |
|---|---|---|---|---|---|---|
| N | 4 | | 4 | | 4 | |
| Cₘₐₓ | 2.46 | (29) | 35.2 | (23) | 368 | (92) |
| (μg/mL) | | | | | | |
| AUC₀₋ₜₗₐₛₜ | 4.81 | (34) | 224 | (29) | 2250 | (51) |
| (μg*day/mL) | | | | | | |
| Tₘₐₓ^{a} | 0.21 | (0.21-0.21) | 0.21 | (0.21-0.21) | 0.21 | (0.21-0.94) |
| (days) | | | | | | |

Abbreviations: AUC₀₋ₜₗₐₛₜ = area under the concentration-time curve from time zero to time t, where t is the last time point with a measurable concentration; Cₘₐₓ = maximum serum concentration; IV = intravenous; N = number of subjects in dose group; Study TGAB = Study I5V-MC-TGAB; Tmax = time to maximum concentration.
^{a}Median (range).

TABLE 6.3
Summary of Noncompartmental Antibody I Pharmacokinetic
Parameters after 5 Q3W IV Doses of Antibody I in Diabetic
Nephropathy Patients in Study TGAB (Part B)

| | Antibody I 50 mg IV | | Antibody I 250 mg IV | | Antibody I 750 mg IV | |
|---|---|---|---|---|---|---|
| N | 8 | | 10 | | 10 | |
| Cₘₐₓ | 20.7 | (37) | 86.5 | (56) | 271 | (69) |
| (μg/mL) | | | | | | |
| Tₘₐₓ^{a} | 0.04 | (0.04-0.08) | 0.04 | (0.0-0.04) | 0.04 | (0.00-15) |
| (days) | | | | | | |
| AUC₀₋ₜₗₐₛₜ | 97.7 | (73) | 1270 | (42) | 4880 | (43) |
| (μg*day/mL) | | | | | | |
| AUC_{τ} | 110^{b} | (52) | 942 | (38) | 3220 | (49) |
| (μg · day/mL) | | | | | | |

Abbreviations: AUC₀₋ₜₗₐₛₜ = area under the concentration-time curve from time zero to time t, where t is the last time point with a measurable concentration; AUC_{τ} = area under the concentration versus time curve during one dosing interval; Cₘₐₓ = maximum serum concentration; IV = intravenous; N = number of subjects in dose group; Q3W = every 3 weeks; Study TGAB = Study I5V-MC-TGAB; Tₘₐₓ = time to maximum concentration.
^{a}Median (range).
^{b}N = 4.

### Pharmacodynamics

### Target Engagement Via Measurement of Total Epiregulin and Total TGFα Concentrations:

Drug-tolerant epiregulin and TGFα assays were used in the clinical program to measure total epiregulin concentrations (total epiregulin or TGFα=Antibody I-bound epiregulin or TGFα+free epiregulin or TGFα). When Antibody I is administered, the premise is that epiregulin/TGFα will bind to Antibody I, and the amount of free epiregulin/TGFα that is available to interact with the EGFR will be reduced. Antibody I-bound epiregulin TGFα is expected to have a lower clearance than free epiregulin/TGFα, resulting in an increase in total epiregulin/TGFα concentration upon Antibody I administration.

Upon administration of Antibody I, serum total epiregulin levels increased in a dose-dependent manner in healthy subjects and patients with DN. Total serum epiregulin levels typically peaked between 1 and 4 weeks after single-dose Antibody I administration and continued to accumulate after subsequent doses. This is consistent with sequestration of total epiregulin in the serum compartment, along with a reduction in its clearance, and hence progressive accumulation of its serum concentration. This is consistent with target engagement to epiregulin.

Serum TGFα levels did not appear to change upon single-dose IV administration of Antibody I in healthy subjects; dose-dependent increases in serum total TGFα concentration were observed after multiple doses in patients with DN. However, the magnitude of serum total TGFα increase was smaller than that of epiregulin, and it was more variable.

Data for total serum epiregulin and TGFα suggest that Antibody I binds to both epiregulin and TGFα and support preclinical data that Antibody I can engage both epiregulin and TGFα in humans in vivo.

### Summary of Safety Data Across Clinical Studies

Study TGAA: No serious adverse events (SAEs) related to Antibody I dosing occurred in this study. No subject withdrew or was discontinued due to an AE considered related to Antibody I. Treatment-emergent Aes (TEAEs) of all causality that were reported by at least 2 subjects (or ≥5%) after receiving Antibody I and were reported more frequently than subjects receiving placebo included rhinitis, sneezing, back pain, cough, feeling hot, paraesthesia, and rhinorrhea. None of the TEAEs that were considered related to study drug were observed in more than 2 subjects randomized to Antibody I. One subject experienced diffuse pruritic maculopapular rash of moderate severity 12 days after dosing with 1 mg of Antibody I. The rash persisted for 10 days and resolved spontaneously. The investigator considered the rash related to study drug dosing and consistent with rash usually associated with the administration of pan-EGFR inhibitors. There were no clinically significant abnormalities of blood pressure, heart rate, QTc, clinical laboratory tests, or lung diffusing capacity.

### Study TGAB:

In Part A, no patients experienced a TEAE that resulted in withdrawal of study drug, an SAE, or death during the study. Treatment-emergent Aes were experienced by 1 of 3 patients who received placebo, 3 of 4 patients who received 10 mg Antibody I, 4 of 4 patients who received 100 mg, and 2 of 4 patients who received 750 mg. One patient in each treatment group had at least 1 drug-related TEAE.

In Part B, 1 patient who received placebo died during the study. The number of patients who experienced at least 1 SAE included 1 of 6 patients who received placebo (16.7%), 3 of 14 patients in the 50-mg group (21.4%), 6 of 13 patients in the 250-mg group (46.2%), and 1 of 12 patients in the 750-mg group (8.3%). A total of 25.6% of patients who received Antibody I in Part B reported an SAE. None of the SAEs observed in the study were judged to be related to study drug dosing, and no SAE was experienced by more than 1 patient, regardless of treatment.

The number of patients who experienced a TEAE that resulted in withdrawal of study drug included 1 of 6 patients who received placebo (16.7%), 2 of 14 patients in the 50-mg group (14.3%), 3 of 13 patients in the 250-mg group (23.1%), and 1 of 12 patients in the 750-mg group (8.3%). With a single exception, all TEAEs that led to withdrawal of study drug were considered unrelated to study drug. The 1 related TEAE that led to withdrawal of study drug was generalized itching in a patient who received 750-mg Antibody I.

The 5 most common TEAEs experienced in Part B by Antibody I-treated patients were diarrhea, edema aggravated, headache, hypertension worsened, and nausea (10.3% each). The 2 most common TEAEs experienced by patients who received placebo were headache and chronic kidney disease (33.3% each). There was no evidence of a dose relationship for the frequency of TEAEs and Antibody I. Treatment-emergent Aes from Study TGAB Part B (regardless of causality) are summarized in Table 6.4. None of the drug-related TEAEs were reported by more than 1 patient in Part B. There were no consistent changes from baseline over time for vital sign values or laboratory values.

TABLE 6.4
Summary of Treatment-Emergent Adverse Events by Descending Frequency
of Preferred Term for All Antibody I - Part B (Safety Population)

| | | | Antibody I | | Antibody I | | Antibody I | | All | |
|---|---|---|---|---|---|---|---|---|---|---|
| | Placebo | | 50 mg | | 250 mg | | 750 mg | | Antibody I | |
| Preferred | (N = 6) | | (N = 14) | | (N = 13) | | (N = 12) | | (N = 39) | |
| Term | n (%) | | n (%) | | n (%) | | n (%) | | n (%) | |
| Any Event | 6 | (100.0) | 13 | (92.9) | 13 | (100.0) | 11 | (91.7) | 37 | (94.9) |
| Diarrhea | 0 | | 1 | (7.1) | 1 | (7.7) | 2 | (16.7) | 4 | (10.3) |
| Edema aggravated | 0 | | 3 | (21.4) | 1 | (7.7) | 0 | | 4 | (10.3) |
| Headache | 2 | (33.3) | 1 | (7.1) | 2 | (15.4) | 1 | (8.3) | 4 | (10.3) |
| Hypertension | 1 | (16.7) | 1 | (7.1) | 2 | (15.4) | 1 | (8.3) | 4 | (10.3) |
| worsened | | | | | | | | | | |
| Nausea | 1 | (16.7) | 1 | (7.1) | 2 | (15.4) | 1 | (8.3) | 4 | (10.3) |

| | | | | | | | | | |
|---|---|---|---|---|---|---|---|---|---|
| Upper respiratory | 1 | (16.7) | 2 | (14.3) | 0 | 1 | (8.3) | 3 | (7.7) |
| infection | | | | | | | | | |

| Acne | 0 | 0 | | 2 | (15.4) | 0 | 2 | (5.1) |
|---|---|---|---|---|---|---|---|---|
| Acute kidney injury | 0 | 1 | (7.1) | 1 | (7.7) | 0 | 2 | (5.1) |
| Anemia aggravated | 0 | 1 | (7.1) | 1 | (7.7) | 0 | 2 | (5.1) |

| | | | | | | | | |
|---|---|---|---|---|---|---|---|---|
| Blood creatinine | 0 | 1 | (7.1) | 0 | 1 | (8.3) | 2 | (5.1) |
| increased | | | | | | | | |

| | | | | | | | | |
|---|---|---|---|---|---|---|---|---|
| Bradycardia | 0 | 0 | 1 | (7.7) | 1 | (8.3) | 2 | (5.1) |
| Bronchitis | 0 | 0 | 1 | (7.7) | 1 | (8.3) | 2 | (5.1) |

| | | | | | | | | |
|---|---|---|---|---|---|---|---|---|
| Cellulitis of leg | 0 | 1 | (7.1) | 0 | 1 | (8.3) | 2 | (5.1) |

| | | | | | | | |
|---|---|---|---|---|---|---|---|
| Dyspnea | 0 | 0 | 2 | (15.4) | 0 | 2 | (5.1) |

| Fatigue aggravated | 0 | 0 | | 0 | 2 | (16.7) | 2 | (5.1) |
|---|---|---|---|---|---|---|---|---|
| Gout flare | 0 | 0 | | 0 | 2 | (16.7) | 2 | (5.1) |
| Hypokalemia | 0 | 1 | (7.1) | 0 | 1 | (8.3) | 2 | (5.1) |

| | | | | | | | |
|---|---|---|---|---|---|---|---|
| Itching | 0 | 0 | 0 | 2 | (16.7) | 2 | (5.1) |

| | | | | | | | | |
|---|---|---|---|---|---|---|---|---|
| Lightheadedness | 0 | 1 | (7.1) | 1 | (7.7) | 0 | 2 | (5.1) |

| Muscle cramps | 1 | (16.7) | 2 | (14.3) | 0 | 0 | | 2 | (5.1) |
|---|---|---|---|---|---|---|---|---|---|
| Orthostatic | 0 | | 2 | (14.3) | 0 | 0 | | 2 | (5.1) |
| hypotension | | | | | | | | | |
| Urinary tract | 0 | | 2 | (14.3) | 0 | 0 | | 2 | (5.1) |
| infection | | | | | | | | | |
| Viral infection | 1 | (16.7) | 1 | (7.1) | 0 | 1 | (8.3) | 2 | (5.1) |

| Volume overload | 0 | | 2 | (14.3) | 0 | | 0 | 2 | (5.1) |
|---|---|---|---|---|---|---|---|---|---|
| Vomiting | 1 | (16.7) | 1 | (7.1) | 1 | (7.7) | 0 | 2 | (5.1) |

| | | | | | | | |
|---|---|---|---|---|---|---|---|
| Wound | 0 | 2 | (14.3) | 0 | 0 | 2 | (5.1) |

Note:
Aes were coded using MeDRA version 17.0. A TEAE was any untoward medical occurrence that either occurred or worsened at any time after treatment baseline (ie, with onset after the first dose of study medication) and which did not necessarily have a causal relationship with this treatment.
Abbreviations: AE = adverse event; MedDRA = Medical Dictionary for Regulatory Activities; N = number of subjects in dose group; TEAE = treatment-emergent AE.

### Example 7: Randomized, Placebo-Controlled, Phase 2 Clinical Trial to Evaluate Antibody I for the Treatment of Osteoarthritis

The purpose of this study is to provide human clinical evidence for Antibody I efficacy in relieving knee pain due to osteoarthritis (OA). Data will be collected to assess the safety, and tolerability of Antibody I in this study population. Pharmacokinetic (PK) properties, pharmacodynamic (PD) effects and immunogenicity profile will also be explored. The totality of data from this proof-of-concept study will assess the benefits and risks associated with Antibody I and inform the clinical development of Antibody I.

### Overall Design:

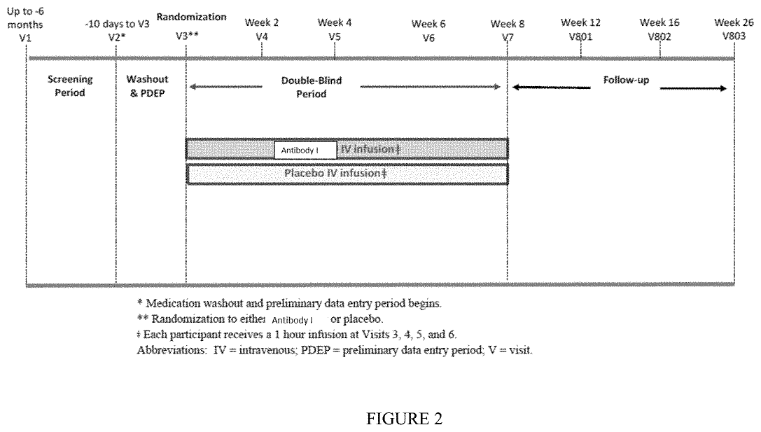

This is a 26-week, Phase 2, randomized, double-blind, placebo-controlled study that will compare Antibody I versus placebo in participants with OA in the knee. This is a randomized, investigator- and participant-blind, placebo controlled, Phase 2 clinical trial. Approximately 125 participants will be randomly assigned to study intervention (84 Antibody I and 41 placebo). This 26-week study includes an 8-week double-blind treatment period and an 18-week follow-up period. This study design is shown in FIG. 2.

The study intervention will be administered via a slow intravenous (IV) infusion over approximately 1 hour by blinded site personnel. The infusion rate may be reduced as deemed necessary if an infusion reaction is observed. The dose is a 750-mg starting dose followed by 500 mg every 2 weeks IV for a total of 4 doses. Dose formulation is a lyophilized powder reconstituted with sterile water 0.9% Sodium chloride solution. Participants will receive an IV infusion every 2 weeks for a total of 4 infusions. Participants will be monitored for at least 4 hours after completion of each infusion.

At patient visits, all post-treatment sample collection and safety monitoring are completed, and participants are instructed to continue with study restrictions and Numeric Rating Scale (NRS) diary entries before their visit discharge. Efficacy data will be collected up to 6 weeks after the last dose, based on the long PK half-life and potential sustained target engagement of Antibody I. Safety, pharmacokinetic (PK), pharmacodynamic (PD) and immunogenicity samples will be collected up to 20 weeks after the last administration of intervention to characterize the safety and clinical immunogenicity profile. A participant is considered to have completed this study if he or she has completed all required phases of the study including the last scheduled procedure.

### Objectives and Endpoints:

Primary and secondary objectives and endpoints are described below.

| Core Assessment | Measure |
|---|---|
| Pain intensity | Visual Analog Scale (VAS) for pain |
| Pain intensity | Numeric Rating Scale (NRS) for pain |
| Participant ratings | Patient Global Impression of Change (PGI) |
| of overall improvement | |
| Emotional functioning | EuroQol-5D 5 level questionnaire (EQ-5D-5L) |
| Quality of sleep | Medical Outcomes Study (MOS) Sleep |
| | Scale |
| Physical functioning | Change from baseline to endpoint |
| | for Western Ontario and McMaster |
| | University Arthritis Index |
| | (WOMAC ®) for pain, stiffness, and |
| | physical function subscales |
| | Proportion of participants with |
| | reduction from baseline greater than |
| | 30%, 50%, and 70% on WOMAC |
| | pain subscale across all time points |
| | Proportion of participants with |
| | reduction from baseline greater than |
| | 30%, 50%, and 70% on WOMAC |
| | physical function subscale across all |
| | time points |

Visual Analog Scale (VAS): The VAS for pain will be used at screening and at each clinic visit. This is a graphic, single-item scale where participants are asked to describe their pain intensity over the past week, in the area under study, on a scale of 0 to 100:0=no pain, and 100=worst imaginable pain. Participants complete the VAS by placing a line perpendicular to the VAS line at a point that describes their pain intensity.

Numeric Rating Scale (NRS): The NRS will be used during the preliminary data entry period (PDEP) and daily throughout the study to describe pain severity. This is a numeric, single-item scale where participants are asked to describe their average and worst pain over the past 24 hours, on a scale of 0 to 10:0=no pain, and 10=pain as bad as you can imagine. Participants complete the NRS daily using a take-home device. Participant compliance is reviewed at each clinic visit. The NRS worst pain will be collected daily. The scores for the other secondary measures will be collected at each visit as specified in the visit schedule. The primary outcome measure is the mean change from baseline to endpoint for average pain intensity as assessed by the NRS item ‘Please rate your pain by selecting the one number [0-10] that describes your AVERAGE level of [area under study] pain during the past 24 hours.’ This measure was selected based on its demonstrated ability to detect changes in pain and its common use across disease states. A secondary measure is the mean change from baseline to endpoint for worst pain intensity as measured by the NRS item ‘Please rate your pain by selecting the one number [0-10] that describes your WORST level of [area under study] pain during the past 24 hours.’ The NRS value of average pain and worst pain over the past 24 hours will be collected daily for each participant. For the statistical analyses, the average NRS value of the average and worst pain over the past 24 hours will be calculated for both weekly intervals and biweekly intervals. The average of the weekly intervals for the NRS will result in 8 postbaseline observations, and the average of the biweekly intervals will result in 4 postbaseline observations for each participant if a participant completes the placebo-controlled portion of the study. The average of the weekly intervals for the NRS will be used in the primary efficacy analysis and other analyses described below, unless otherwise specified in the analysis plan. A participant must have 50% or greater of the daily NRS values during the prespecified time interval to calculate the average NRS value; otherwise, the average NRS value for that visit will be considered missing.

Participant Ratings on Overall Improvement: The Patient Global Impression of Change (PGI) is used across disease states. It captures the participant's perspective of treatment apart from sub-aspects of the general improvement. This is a numeric scale from 1 to 7: 1=very much better, and 7=very much worse. The participant is asked to ‘Mark the box that best describes how your pain symptoms are now, compared to how they were before you started taking this medicine.’

Emotional Functioning Assessments: The EQ-5D-5L health status questionnaire is used across disease states. The EQ-5D-5L is one of the most popular patient-completed instruments to address quality of life (Buchholz I, Janssen M F, Kohlmann T, Feng Y S. A systematic review of studies comparing the measurement properties of the three-level and five-level versions of the EQ-5D. PharmacoEconomics. 2018; 36(6):645-661.). It is a descriptive system that includes 5 dimensions: mobility, self-care, usual activities, pain/discomfort, and anxiety/depression. The participant is asked to ‘check the ONE box that best describes your health TODAY,’ choosing from 5 options provided under each dimension. The scores on the 5 dimensions can be presented as a health profile or converted to a single summary index number. The EQ-5D-5L also includes the EQ VAS, which records the participant's self-rated health on a vertical VAS of 0 to 100:0=the worst health you can imagine, and 100=the best health you can imagine. The instrument used in its EQ-5D-5L version is a short, reliable, validated, easy-to-complete scale with excellent test-retest reliability to address quality of life in relation to pain due to several diseases.

Sleep Quality: Sleep disturbance is an important issue in pain research. Among various available instruments, the Medical Outcome Study (MOS) Sleep Scale provides a unique, psychometrically validated score for sleep disturbance. This scale consists of 12 questions addressing the past week. Participants report how often each sleep symptom or problem is present on a 6-point categorical scale ranging from ‘all of the time’ to ‘none of the time.’ Questions about time to fall asleep and quantity of sleep are reported as the average number of hours slept each night. This scale has low administration burden, has been used in different pain studies, and has been validated in patients with neuropathic pain.

Safety Assessments: Planned time points for safety assessments are determined according to typical practices, and include physical examination, vital sign and body weight measurements, 12-lead ECGs, clinical laboratory tests, hepatic safety monitoring, C-SSRS, and spontaneously reported Aes.

### Efficacy Assessments:

| | |
|---|---|
| Objective | Endpoint Measure |

Primary:

| | |
|---|---|
| Pain Intensity: | Mean change from baseline assessment to |
| Efficacy of Antibody | endpoint for average pain intensity as |
| I versus placebo | measured by numeric rating scale (NRS) |

Secondary:

| | |
|---|---|
| Physical Functioning: | Mean change from baseline assessment to |
| Efficacy of Antibody | endpoint for physical functioning |
| I versus placebo | measures as described in the 24-question |
| | WOMAC and subscales. |
| Overall Improvement: | Mean change from baseline assessment to |
| Efficacy of Antibody | endpoint for overall improvement as |
| I versus placebo | measured by Patient's Global Impression of |
| | Change |
| Other Efficacy: | Mean change from baseline |
| Efficacy of Antibody | assessment to endpoint for worst |
| I versus placebo | pain intensity as measured by NRS |
| | Proportion of patients with a pain |
| | reduction from baseline greater than |
| | 30%, 50%, and 70% as measured by |
| | the average and worst pain |
| | responses on the NRS |
| | Proportion of patients with 2-point |
| | reduction from baseline as |
| | measured by the average and worst |
| | pain responses on the NRS |
| | Time to treatment response with a |
| | 30%, 50%, and 70% reduction from |
| | baseline as measured by the average |
| | and worst pain responses on the |
| | NRS |
| | Time to treatment response with 2- |
| | point reduction from baseline as |
| | measured by the average and worst |
| | pain |
| | Mean change from baseline |
| | assessment to endpoint on the visual |
| | analog scale (VAS) for pain |
| | responses on the NRS |
| | Proportion of patients with pain |
| | reduction from baseline greater than |
| | 30%, 50%, and 70% as measured by |
| | VAS |
| | Mean change from baseline |
| | assessment to endpoint on the Sleep |
| | Scale from the Medical Outcomes |
| | Study (MOS Sleep Scale) |
| | Proportion of patients with |
| | reduction from baseline greater than |
| | 30%, 50%, and 70% on the physical |
| | functioning measures as described |
| | in the WOMAC, and |
| | Summary of frequency, timing, and |
| | amount of rescue medication used |
| | during the treatment phase. |
| Emotional Functioning: | Mean change from baseline assessment to |
| Efficacy of each | endpoint for emotional functioning as |
| study intervention | measured by the EuroQol-5D 5 level |
| versus placebo on | questionnaire (EQ-5D-5L) |
| patient-reported | |
| clinical outcomes | |

A core set of assessments and domains are used when characterizing chronic pain, which are: pain, physical functioning, emotional functioning, participant ratings of overall improvement, adverse events (Aes), and participant disposition. The NRS is selected for the primary endpoint based on its demonstrated ability to detect changes in pain and its common use across the disease states under study.

Western Ontario and McMaster Universities Osteoarthritis Index: The Western Ontario and McMaster Universities Osteoarthritis Index (WOMAC®) (Bellamy N. The WOMAC Knee and Hip Osteoarthritis Indices: Development, validation, globalization and influence on the development of the AUSCAN Hand Osteoarthritis Indices. Clin Exp Rheumatol. 2005; 23(Suppl. 39):148-153) is a validated instrument that is extensively used to evaluate the response to medications for the treatment of osteoarthritic pain. The WOMAC version LK3.1 is administered according to the schedule of activities.

This table describes the 24-question WOMAC and subscales.

| Dimensions | Number of Questions |
|---|---|
| pain | 5 |
| stiffness | 2 |
| physical function | 17 |

The participants will record their responses using a 0 to 4 Likert scale for each question: 0=no pain, and 4=extreme pain. The scores for each subscale will be calculated by summing the scores of the questions in the respective subscale for each participant at each time point. The range of possible scores for each subscale is “pain=0 to 20”, “stiffness=0 to 8”, and “physical function=0 to 68”. A Bayesian longitudinal mixed-model repeated measures analysis (MMRM) will be performed to evaluate the change from baseline to each post baseline visit for the WOMAC pain subscale and physical function subscale.

The proportion of participants in each treatment group meeting pre-specified binary efficacy outcomes will be estimated for each post baseline time point and will be used to compare treatment groups. The estimates will be provided from fitting a Bayesian longitudinal model that includes all post baseline observations. The prespecified binary efficacy outcomes include the proportion of participants within an ISA: with a reduction greater than 30%, 50%, and 70% from baseline as measured by WOMAC pain subscale, and with a reduction greater than 30%, 50%, and 70% from baseline as measured by WOMAC physical function subscale. The model will include the categorical and continuous covariates described in the continuous efficacy analysis model above, except the interaction of baseline and visit will not be used. Additional model terms may be considered and specified in statistical analysis plan. A cumulative distribution function of percent change from baseline to endpoint for the WOMAC pain and physical function subscales will be provided for each treatment group.

### Schedule of Activities (SoA):

This schedule of activities shows certain visits and procedures for a study of Antibody I. “Master protocol” refers a protocol setup to guide several potential studies, in a given disease state or multiple disease states, and intervention specific addendum refers to the part of the protocol specifically related to a given intervention under study.

| | Screening^{a} | | | | | | | | |
|---|---|---|---|---|---|---|---|---|---|
| | | Pre- | | | | | | | |
| | Screening | Randomization | Double-Blind Treatment Period | | | | | | |
| | Visit for | Screening | Randomization | | | | | Early | |
| | Master | Visit | to ISA | | | | | Discontinuation | Notes |
| | Visit Number | | | | | | | | |
| | | | | | | | | | Visits after V7 may |
| | | | | | | | | | be specified in the |
| | V1^{b} | V2^{c} | V3 | V4 | V5 | V6 | V7 | ED | ISA. |
| | Study Week | | | | | | | | |
| HOP-MC-CPMP | | −10 to −7 days for | | | | | | | |
| Master | Up to −6 | most | | | | | | | |
| Protocol | months | participants | 0 | 2 | 4 | 6 | 8 | | |

Scales, Questionnaires, and Outcome Measures

| | | | | | | | | | |
|---|---|---|---|---|---|---|---|---|---|
| VAS for pain | X | X | X | X | X | X | X | X | |
| NRS for pain | | X | X | X | X | X | X | X | Collected daily with a |
| | | | | | | | | | take-home device. |
| | | | | | | | | | Compliance reviewed |
| | | | | | | | | | at each clinic visit. |
| Rescue | | X | X | X | X | X | X | X | Total quantity |
| medication | | | | | | | | | collected daily with a |
| usage reporting | | | | | | | | | take-home device. |
| | | | | | | | | | Compliance reviewed |
| | | | | | | | | | at each clinic visit. |
| Pain | X | | | | | | | | |
| catastrophizing | | | | | | | | | |
| scale | | | | | | | | | |
| PGI | | | | X | X | X | X | X | |
| EQ-5D-5L | | | X | X | X | X | X | X | |
| MOS Sleep | | | X | X | X | X | X | X | |
| Scale | | | | | | | | | |

| | Scrrening^{a} | | | | | | | | |
|---|---|---|---|---|---|---|---|---|---|
| | | Pre- | | | | | | | |
| | | Randomization | | | | | | | |
| | Screening | Screening | Double-Blind Treatment Period | | | | | Early | |
| | Visit | Visit | Randomization | | | | | Discontinuation | Notes |
| | Visit Number | | | | | | | | |
| | V1^{b} | V2^{c} | V3 | V4 | V5 | V6 | V7 | ED | |
| | Study Week | | | | | | | | |
| H0P-MC- | Up to −6 | | | | | | | | |
| OA01 DSA | months | −10 to −7 days | 0 | 2 | 4 | 6 | 8 | | |
| Procedure | | | | | | | | | |
| Confirm DSA | X | X | | | | | | | |
| inclusion and | | | | | | | | | |
| exclusion | | | | | | | | | |
| criteria | | | | | | | | | |
| X-ray | X | | | | | | | | Anterior/Posterior and |
| | | | | | | | | | lateral weight-bearing |
| | | | | | | | | | knee radiographs |
| WOMAC ® | | | X | X | X | X | X | X | |

Abbreviations: DSA = disease-state addendum; ED = early discontinuation; V = visit; WOMAC = Western Ontario and McMaster University Osteoarthritis Index.
^{a}Screening assessments may be conducted at other time points prior to randomization if they reduce participant burden.
^{b}The site determines the half-life of each pain medication the participant is currently taking in order to schedule Visit 2. Visit 2 can be scheduled no earlier than 7 days prior to randomization at Visit 3 due to the required 7-day PDEP
^{c}The 5 half-life washout period for pain medications must come before the PDEP, resulting in a minimum of 10 days for most participants.

### Study Population:

Male and female participants are eligible for inclusion in the study if they have a history of daily pain based on patient report or medical history.

### Inclusion Criteria:

Participants are eligible to be included in the study only if all the following criteria apply:

they are 40 years or older in age at the time of signing the informed consent;

they have presence of index knee pain for >12 weeks at Visit 1;

they have an x-ray supporting diagnosis of osteoarthritis according to the American College of Rheumatology with a Kellgren-Lawrence grade 2 to 4 radiographic classification of index knee; they have stable glycemic control as indicated by a glycated hemoglobin (HbA1c) less than or equal to 10 at time of screening;

they are men or women who abide by the reproductive and contraceptive requirements provided; they are willing to discontinue all pain medications for condition under study except rescue medication permitted until V801 in the follow-up period;

they must have venous access in both arms for IV infusion and sample collection.

### Pain Characteristics:

they have a visual analog scale (VAS) pain value >40 and <95 at Visits 1 and 2;

they have a history of daily pain for at least 12 weeks based on patient report or medical history;

they have a value of ≤30 on the pain catastrophizing scale;

Weight: they have a body mass index <40 kg/m2 (inclusive)

### Informed Consent:

they are capable of providing informed consent, which includes compliance with the requirements and restrictions listed in the informed consent form (ICF) and in the protocol;

they are reliable, willing, and able to participate in all required protocol procedures for the duration of the study;

they are willing to maintain a consistent regimen of any ongoing nonpharmacologic pain-relieving therapies (for example, physical therapy) and will not start any new nonpharmacologic pain-relieving therapies during study participation;

they are willing to discontinue all pain medications for condition under study,

except rescue medication permitted per protocol, for the duration of the study;

they must enter the required daily assessments during the PDEP for at least 5 of the 7 days prior to randomization.

### Exclusion Criteria:

Participants are excluded from the study if any of the following criteria apply:

they are largely or wholly incapacitated and unable to participate fully in all protocol procedures, for example, bedridden or confined to a wheelchair, permitting little or no selfcare;

they have presence of surgical hardware or other foreign body in the index knee;

they have an unstable index joint (such as a torn anterior cruciate ligament);

they have had a surgical procedure or therapeutic injection in the affected knee within 3 months prior to starting the washout period;

they have fibromyalgia, chronic pain syndrome, or other concurrent medical or arthritic conditions that could interfere with the evaluation of the index knee;

they have a history of Reiter's syndrome, rheumatoid arthritis, psoriatic arthritis, ankylosing spondylitis, arthritis associated with inflammatory bowel disease, sarcoidosis, or amyloidosis;

they have clinical signs and symptoms of active knee infection or crystal disease of the index knee;

they have a history of infection in the index joint;

they have a history of arthritis due to crystals (e.g., gout, pseudogout);

they have ipsilateral hip osteoarthritis; or

Other Medical Conditions: they have had an intra-articular injection of hyaluronic acid within 24 weeks of Visit 2; have an eGFR of less than 70 ml/min/1.73m2 based on CKD-EPI formula at Visit 1 or Visit 2; Have any clinically serious or unstable cardiovascular, musculoskeletal disorder, gastrointestinal, endocrinologic, hematologic, hepatic, metabolic, urologic, pulmonary, dermatologic, immunologic, or ophthalmologic disease within 3 months of Visit 3.

Prior/Concomitant Therapy: they have received any antibodies against nerve growth factor (NGF), or antibodies against EGFR, or EGFR tyrosine kinase inhibitors; have a history of allergic reactions to monoclonal antibodies, or clinically significant multiple or severe drug allergies, including but not limited to erythema multiforme major, linear immunoglobulin A dermatosis, toxic epidermal necrolysis, or exfoliative dermatitis; have a history or presence of uncontrolled asthma, eczema, significant atopy, significant hereditary angioedema or common variable immune deficiency, and

Reproductive: they are women who are pregnant or breastfeeding. Participants are required to maintain similar levels of activity during the double-blind study period. Starting a new exercise program or new strenuous activity is not allowed. Participants who receive physical therapy for OA of the index knee should remain on the same therapy program (intensity and frequency).

### Study Assessments and Procedures:

### Pharmacokinetics:

Venous blood samples of approximately 4 mL will be collected for measurement of Antibody I concentrations. Samples will be used to evaluate the PK of Antibody I. Site personnel will record the date and time (24-hour clock time) of Antibody I administration (start and end of infusion), and the date and time (24-hour clock time) of each PK sample.

### Pharmacodynamics:

Venous blood samples of approximately 4 mL each will be collected for the measurement of epiregulin. Any remaining blood samples may be used to test other potential PD endpoints, including, but not limited to TGF-α.

### Immunogenicity Assessments:

Immunogenicity will be assessed by a validated assay designed to detect ADAs in the presence of Antibody I at a laboratory approved by the sponsor. Antibodies may be further characterized for their ability to neutralize the activity of Antibody I. At the visits and times specified, predose venous blood samples will be collected to determine antibody production against Antibody I. The actual date and time (24-hour clock time) of each sample collection will be recorded. If the immunogenicity sample at the last scheduled assessment or discontinuation visit is treatment-emergent (TE) anti-drug antibody (ADA) positive, additional samples may be taken until the signal returns to baseline (i.e., no longer TE-ADA positive) or for up to 1 year after last dose. To aid interpretation of these results, a predose blood sample for PK analysis will be collected at the same time points.

### Statistical Hypotheses:

A Bayesian critical success factor (CSF) is defined and used to evaluate whether Antibody I met its primary endpoint. The CSF will be evaluated for the primary efficacy endpoint, average pain intensity as measured by the NRS, using the methodology described herein and known to the skilled artisan, and will be calculated at the conclusion of the double-blind portion of each study. The CSF will have the general form of: probability (treatment effect<effect of interest)>probability threshold. The treatment effect will be defined as the Antibody I estimate-placebo estimate of the change from baseline at endpoint. The effect of interest is typically found through a literature search or clinical judgement. The probability threshold is generally set to have a desired level of confidence in the treatment effect or to have the desired operating characteristics under a range of plausible, assumed drug effect scenarios of truth, including a null effect. Additional hypotheses will include the comparison of the active intervention with placebo for the prespecified objectives and endpoints defined herein. The study may be conducted in a protocol wherein multiple studies are contemplated and placebo data may be shared as appropriate.

The decision criterion for the primary hypothesis is defined as being at least 70% confident that Antibody I is at least 0.55 units better than placebo on average pain intensity as measured by the NRS. The key secondary null hypothesis is that there is no difference between Antibody I and placebo on the key secondary endpoint, the mean change from baseline to endpoint for the WOMAC® Pain Subscale Score. The decision criterion for the key secondary hypothesis is defined as being at least 70% confident that Antibody I is at least 0.35 units better than placebo on the WOMAC® Pain Subscale.

### Sample Size Determination:

Approximately 125 participants will be randomized in a 2:1 ratio to Antibody I and placebo, respectively. It is expected that approximately 107 participants will complete the double-blind treatment period of the study. This sample size will provide greater than 80% power to demonstrate that the active treatment arm has a ≥0.7 posterior probability of being better than placebo by at least 0.35 units on the WOMAC® Pain Subscale (WOMAC=Western Ontario and McMaster University Osteoarthritis Index).

The assumption for the power calculation is that mean reductions in pain intensity from baseline, as measured by the WOMAC Pain Subscale, are approximately 3 units and 4 units at endpoint, for placebo and Antibody I, respectively, with a common standard deviation of 2.25. If there is no treatment difference between placebo and Antibody I, the probability of passing the efficacy criterion specified above (i.e., false positive) is approximately 0.1. The simulation for the power calculation and sample size determination was carried out in FACTS Version 6.0.

### Populations for Analyses:

The pharmacokinetic population includes for example all randomized participants who received a full dose of Antibody I at Visit 3 and have at least 1 evaluable PK sample collected prior to dosing at or after Visit 4.

### Example 8: Randomized, Placebo-Controlled, Phase 2 Clinical Trial to Evaluate Antibody I for the Treatment of Diabetic Peripheral Neuropathic Pain

The purpose of this study is to provide human clinical evidence of Antibody I efficacy in relieving diabetic peripheral neuropathic pain (DPNP). Data will be collected to assess the safety, and tolerability of Antibody I in this study population. Pharmacokinetic (PK) properties, pharmacodynamic (PD) effects and immunogenicity profile will also be explored. The totality of data from this proof-of-concept study will assess the benefits and risks associated with Antibody I and inform the clinical development of Antibody I.

### Overall Design:

This is a 26-week, Phase 2, randomized, double-blind, placebo-controlled study that will compare Antibody I versus placebo in participants with DPNP. This is a randomized, investigator- and participant-blind, placebo controlled, Phase 2 clinical trial. Approximately 125 participants will be randomly assigned to study intervention (84 Antibody I and 41 placebo). This 26-week study includes an 8-week double-blind treatment period and an 18-week follow-up period. This study design is shown in FIG. 2.

The study intervention will be administered via a slow intravenous (IV) infusion over approximately 1 hour by blinded site personnel. The infusion rate may be reduced as deemed necessary if an infusion reaction is observed. The dose is a 750-mg starting dose followed by 500 mg every 2 weeks IV for a total of 4 doses. Dose formulation is a lyophilized powder reconstituted with sterile water 0.9% Sodium chloride solution. Participants will receive an IV infusion every 2 weeks for a total of 4 infusions. Participants will be monitored for at least 4 hours after completion of each infusion.

At patient visits, all post-treatment sample collection and safety monitoring are completed, and participants are instructed to continue with study restrictions and Numeric Rating Scale (NRS) diary entries before their visit discharge. Efficacy data will be collected up to 6 weeks after the last dose, based on the long PK half-life and potential sustained target engagement of Antibody I. Safety, pharmacokinetic (PK), pharmacodynamic (PD) and immunogenicity samples will be collected up to 20 weeks after the last administration of intervention to characterize the safety and clinical immunogenicity profile. A participant is considered to have completed this study if he or she has completed all required phases of the study including the last scheduled procedure.

### Objectives and Endpoints:

Primary and secondary objectives and endpoints are described below.

| Core Assessment | Measure |
|---|---|
| Pain intensity | Visual Analog Scale (VAS) for pain |
| Pain intensity | Numeric Rating Scale (NRS) for pain |
| Participant ratings | Patient Global Impression of Change (PGI) |
| of overall improvement | |
| Emotional functioning | EuroQol-5D 5 level questionnaire (EQ-5D-5L) |
| Quality of sleep | Medical Outcomes Study (MOS) Sleep |
| | Scale |
| Physical functioning | Change from baseline to endpoint |
| | for the Brief Pain Inventory- |
| | Short Form (BPI-SF) for the: |
| | Individual severity score, |
| | Individual interference score, |
| | Total interference score. |
| | Proportion of participants with |
| | reduction from baseline >30%, |
| | 50%, and 70% on the BPI-SF |
| | individual severity scores. |
| | Proportion of participants with |
| | reduction from baseline >30%, |
| | 50%, and 70% on the BPI-SF total |
| | interference score. |

Visual Analog Scale (VAS): The VAS for pain will be used at screening and at each clinic visit. This is a graphic, single-item scale where participants are asked to describe their pain intensity over the past week, in the area under study, on a scale of 0 to 100:0=no pain, and 100=worst imaginable pain. Participants complete the VAS by placing a line perpendicular to the VAS line at a point that describes their pain intensity.

Numeric Rating Scale (NRS): The NRS will be used during the preliminary data entry period (PDEP) and daily throughout the study to describe pain severity. This is a numeric, single-item scale where participants are asked to describe their average and worst pain over the past 24 hours, on a scale of 0 to 10:0=no pain, and 10=pain as bad as you can imagine. Participants complete the NRS daily using a take-home device. Participant compliance is reviewed at each clinic visit. The NRS worst pain will be collected daily. The scores for the other secondary measures will be collected at each visit as specified in the visit schedule. The primary outcome measure is the mean change from baseline to endpoint for average pain intensity as assessed by the NRS item ‘Please rate your pain by selecting the one number [0-10] that describes your AVERAGE level of [area under study] pain during the past 24 hours.’ This measure was selected based on its demonstrated ability to detect changes in pain and its common use across disease states. A secondary measure is the mean change from baseline to endpoint for worst pain intensity as measured by the NRS item ‘Please rate your pain by selecting the one number [0-10] that describes your WORST level of [area under study] pain during the past 24 hours.’ The NRS value of average pain and worst pain over the past 24 hours will be collected daily for each participant. For the statistical analyses, the average NRS value of the average and worst pain over the past 24 hours will be calculated for both weekly intervals and biweekly intervals. The average of the weekly intervals for the NRS will result in 8 postbaseline observations, and the average of the biweekly intervals will result in 4 postbaseline observations for each participant if a participant completes the placebo-controlled portion of the study. The average of the weekly intervals for the NRS will be used in the primary efficacy analysis and other analyses described below, unless otherwise specified in the analysis plan. A participant must have 50% or greater of the daily NRS values during the prespecified time interval to calculate the average NRS value; otherwise, the average NRS value for that visit will be considered missing.

Participant Ratings on Overall Improvement: The Patient Global Impression of Change (PGI) is used across disease states. It captures the participant's perspective of treatment apart from sub-aspects of the general improvement. This is a numeric scale from 1 to 7:1=very much better, and 7=very much worse. The participant is asked to ‘Mark the box that best describes how your pain symptoms are now, compared to how they were before you started taking this medicine.’

Emotional Functioning Assessments: The EQ-5D-5L health status questionnaire is used across disease states. The EQ-5D-5L is one of the most popular patient-completed instruments to address quality of life (Buchholz I, Janssen M F, Kohlmann T, Feng Y S. A systematic review of studies comparing the measurement properties of the three-level and five-level versions of the EQ-5D. PharmacoEconomics. 2018; 36(6):645-661.). It is a descriptive system that includes 5 dimensions: mobility, self-care, usual activities, pain/discomfort, and anxiety/depression. The participant is asked to ‘check the ONE box that best describes your health TODAY,’ choosing from 5 options provided under each dimension. The scores on the 5 dimensions can be presented as a health profile or converted to a single summary index number. The EQ-5D-5L also includes the EQ VAS, which records the participant's self-rated health on a vertical VAS of 0 to 100:0=the worst health you can imagine, and 100=the best health you can imagine. The instrument used in its EQ-5D-5L version is a short, reliable, validated, easy-to-complete scale with excellent test-retest reliability to address quality of life in relation to pain due to several diseases.

Sleep Quality: Sleep disturbance is an important issue in pain research. Among various available instruments, the Medical Outcome Study (MOS) Sleep Scale provides a unique, psychometrically validated score for sleep disturbance. This scale consists of 12 questions addressing the past week. Participants report how often each sleep symptom or problem is present on a 6-point categorical scale ranging from ‘all of the time’ to ‘none of the time.’ Questions about time to fall asleep and quantity of sleep are reported as the average number of hours slept each night. This scale has low administration burden, has been used in different pain studies, and has been validated in patients with neuropathic pain.

Safety Assessments: Planned time points for safety assessments are determined according to typical practices, and include physical examination, vital sign and body weight measurements, 12-lead ECGs, clinical laboratory tests, hepatic safety monitoring, C-SSRS, and spontaneously reported Aes.

### Efficacy Assessments:

| | |
|---|---|
| Objective | Endpoint Measure |

Primary:

| | |
|---|---|
| Pain Intensity: | Mean change from baseline assessment to |
| Efficacy of Antibody | endpoint for average pain intensity as |
| I versus placebo | measured by numeric rating scale (NRS) |

Secondary:

| | |
|---|---|
| Physical Functioning: | Mean change from baseline assessment to |
| Efficacy of Antibody | endpoint for physical functioning |
| I versus placebo | measures as described in: |
| | Change from baseline to endpoint |
| | for the Brief Pain Inventory- |
| | Short Form (BPI-SF) for the: |
| | Individual severity score, |
| | Individual interference score, |
| | Total interference score. |
| | Proportion of participants with |
| | reduction from baseline >30%, 50%, and |
| | 70% on the BPI-SF individual severity |
| | scores. |
| | Proportion of participants with |
| | reduction from baseline >30%, |
| | 50%, and 70% on the BPI-SF total |
| | interference score. |
| Overall Improvement: | Mean change from baseline assessment to |
| Efficacy of Antibody | endpoint for overall improvement as |
| I versus placebo | measured by Patient's Global Impression of |
| | Change |
| Other Efficacy: | Mean change from baseline |
| Efficacy of Antibody | assessment to endpoint for worst |
| I versus placebo | pain intensity as measured by NRS |
| | Proportion of patients with a pain |
| | reduction from baseline greater than |
| | 30%, 50%, and 70% as measured by |
| | the average and worst pain |
| | responses on the NRS |
| | Proportion of patients with 2-point |
| | reduction from baseline as |
| | measured by the average and worst |
| | pain responses on the NRS |
| | Time to treatment response with a |
| | 30%, 50%, and 70% reduction from |
| | baseline as measured by the average |
| | and worst pain responses on the |
| | NRS |
| | Time to treatment response with 2- |
| | point reduction from baseline as |
| | measured by the average and worst |
| | pain |
| | Mean change from baseline |
| | assessment to endpoint on the visual |
| | analog scale (VAS) for pain |
| | responses on the NRS |
| | Proportion of patients with pain |
| | reduction from baseline greater than |
| | 30%, 50%, and 70% as measured by |
| | VAS |
| | Mean change from baseline |
| | assessment to endpoint on the Sleep |
| | Scale from the Medical Outcomes |
| | Study (MOS Sleep Scale) |
| | Proportion of patients with |
| | reduction from baseline greater than |
| | 30%, 50%, and 70% on the physical |
| | functioning measures as described |
| | in the Brief Pain Inventory- |
| | Modified Short Form (BPI-SF), and |
| | Summary of frequency, timing, and |
| | amount of rescue medication used |
| | during the treatment phase. |
| Emotional Functioning: | Mean change from baseline assessment to |
| Efficacy of each | endpoint for emotional functioning as |
| study intervention | measured by the EuroQol-5D 5 level |
| versus placebo on | questionnaire (EQ-5D-5L) |
| patient-reported | |
| clinical outcomes | |

A core set of assessments and domains are used when characterizing chronic pain, which are: pain, physical functioning, emotional functioning, participant ratings of overall improvement, adverse events (Aes), and participant disposition. The NRS is selected for the primary endpoint based on its demonstrated ability to detect changes in pain and its common use across the disease states under study.

### Brief Pain Inventory—Modified Short Form (BPI-SF)

The BPI-SF is a numeric rating scale that assesses the severity of pain (severity scale), its impact on daily functioning (Interference scale), and other aspects of pain (for example, location of pain, relief from medications) in various disease states (Cleeland C S, Ryan K M. Pain assessment: global use of the Brief Pain Inventory. Ann Acad Med Singapore. 1994 March; 23(2):129-138). This table describes the pain scales and corresponding numeric rating scale used in a modified version of the BPI, validated for pain in diabetic polyneuropathy. Participants will rate their pain severity and how, during the past 24 hours, the pain has interfered with the activities described in this table.

| Assessment | Topic | Numeric Rating Scale 0-10 |
|---|---|---|
| 4-item Pain | Worst pain in last 24 | 0 = no pain |
| severity | hours | 10 = pain as |
| | Least pain in the last 24 | bad as you can |
| | hours | imagine |
| | Average pain | |
| | Pain right now | |
| 7-item Pain | General Activity | 0 = does not |
| interference | Mood | interfere |
| | Walking ability | 10 = completely |
| | Normal work | interferes |
| | Relations with | |
| | others | |
| | Sleep | |
| | Enjoyment of life | |

### Schedule of Activities (SoA):

This schedule of activities shows certain visits and procedures for a study of Antibody I. “Master protocol” refers a protocol setup to guide several potential studies, in a given disease state or multiple disease states, disease state addendum refers to guidance for a specific disease state, and intervention specific addendum refers to the part of the protocol specifically related to a given intervention under study.

| | Screening^{a} | | | | | | | | |
|---|---|---|---|---|---|---|---|---|---|
| | | Pre- | | | | | | | |
| | Screening | Randomization | Double-Blind Treatment Period | | | | | | |
| | Visit for | Screening | Randomization | | | | | Early | |
| | Master | Visit | to ISA | | | | | Discontinuation | Notes |
| | Visit Number | | | | | | | | |
| | | | | | | | | | Visits after V7 |
| | | | | | | | | | may be specified in |
| | V1^{b} | V2^{c} | V3 | V4 | V5 | V6 | V7 | ED | the ISA. |
| HOP-MC- | Study Week | | | | | | | | |
| CPMP | | −10 to −7 days | | | | | | | |
| Master | Up to −6 | for most | | | | | | | |
| Protocol | months | participants | 0 | 2 | 4 | 6 | 8 | | |
| VAS for pain | X | X | X | X | X | X | X | X | |

Scales, Questionnaires, and Outcome Measures

| | | | | | | | | | |
|---|---|---|---|---|---|---|---|---|---|
| NRS for pain | | X | X | X | X | X | X | X | Collected daily with |
| | | | | | | | | | a take-home device. |
| | | | | | | | | | Compliance |
| | | | | | | | | | reviewed at each |
| | | | | | | | | | clinic visit. |
| Rescue | | X | X | X | X | X | X | X | Total quantity |
| medication | | | | | | | | | collected daily with |
| usage | | | | | | | | | a take-home device. |
| reporting | | | | | | | | | Compliance |
| | | | | | | | | | reviewed at each |
| | | | | | | | | | clinic visit. |
| Pain | X | | | | | | | | |
| catastrophizing | | | | | | | | | |
| scale | | | | | | | | | |
| PGI | | | | X | X | X | X | X | |
| EQ-5D-5L | | | X | X | X | X | X | X | |
| MOS Sleep | | | X | X | X | X | X | X | |
| Scale | | | | | | | | | |

| | Screening^{a} | | | | | | | | |
|---|---|---|---|---|---|---|---|---|---|
| | | Pre- | | | | | | | |
| | | Randomization | | | | | | | |
| | Screening | Screening | Double-Blind Treatment Period | | | | | Early | |
| | Visit | Visit | Randomization | | | | | Discontinuation | Notes |
| | Visit Number | | | | | | | | |
| | V1^{b} | V2^{c} | V3 | V4 | V5 | V6 | V7 | ED | |
| H0P-MC- | Study Week | | | | | | | | |
| NP03 DSA | | | | 2 | 4 | 6 | 8 | | |
| Procedure | | | | | | | | | |
| Confirm DSA | X | X | | | | | | | |
| inclusion and | | | | | | | | | |
| exclusion | | | | | | | | | |
| criteria | | | | | | | | | |
| Michigan | X | | | | | | | | |
| Neuropathy | | | | | | | | | |
| Screening | | | | | | | | | |
| Instrument | | | | | | | | | |
| Brief pain | | | X | X | X | X | X | X | |
| inventory- | | | | | | | | | |
| modified | | | | | | | | | |
| short form | | | | | | | | | |

Abbreviations: DSA = disease-state addendum; ED = early discontinuation; V = visit.
^{a}Screening assessments may be conducted at other time points prior to randomization if they reduce participant burden.
^{b}The site determines the half-life of each pain medication the participant is currently taking in order to schedule Visit 2. Visit 2 can be scheduled no earlier than 7 days prior to randomization at Visit 3 due to the required 7 -day PDEP
^{c}The 5 half-life washout period for pain medications must come before the PDEP, resulting in a minimum of 10 days for most participants.

### Study Population:

Male and female participants are eligible for inclusion in the study if they have a history of daily pain based on patient report or medical history. Estimated GFR will be used for stratification to ensure a balanced number of participants for analysis of renal effects. Measurements will be collected at Visit 2 and the subsets are greater than or equal to 90, and less than 90.

The Michigan Neuropathy Screening Instrument is used to assess neuropathy in the legs and feet of patients with diabetes (The Michigan Neuropathy Screening Instrument. University of Michigan web site. Available at: http://www.med.umich.edu/borc/profs/documents/svi/MNSI_patient.pdf. Published 2000. Accessed Dec. 11, 2019.). It will be administered according to the schedule of activities. This table describes the assessments included in the instrument.

| Part | Assessment | Completed by | Number of Items |
|---|---|---|---|
| A | History | Study participant | 15 |
| B | Physical | Health professional | 5 |

Both Part A and Part B will be administered. Only Part B will be used to determine inclusion into the study.

### Inclusion Criteria:

Participants are eligible to be included in the study only if all the following criteria apply:

they are 18 years or older in age at the time of signing the informed consent;

they have daily symmetrical foot pain secondary to peripheral neuropathy present for at least 6 months and as diagnosed through use of the Michigan Neuropathy Screening Instrument Part B≥3 (©University of Michigan [WWW]);

they have a history and current diagnosis of type 1 or type 2 diabetes mellitus;

they have stable glycemic control as indicated by a glycated hemoglobin ≤11 at time of screening;

they are men or women who abide by the reproductive and contraceptive requirements provided;

they are willing to discontinue all pain medications for condition under study except rescue medication permitted until V801 in the follow-up period;

they must have venous access in both arms for IV infusion and sample collection.

### Pain Characteristics:

they have a visual analog scale (VAS) pain value >40 and <95 at Visits 1 and 2;

they have a history of daily pain for at least 12 weeks based on patient report or medical history;

they have a value of ≤30 on the pain catastrophizing scale;

Weight: they have a body mass index <40 kg/m2 (inclusive).

### Informed Consent:

they are capable of providing informed consent, which includes compliance with the requirements and restrictions listed in the informed consent form (ICF) and in the protocol;

they are reliable, willing, and able to participate in all required protocol procedures for the duration of the study;

they are willing to maintain a consistent regimen of any ongoing nonpharmacologic pain-relieving therapies (for example, physical therapy) and will not start any new nonpharmacologic pain-relieving therapies during study participation;

they are willing to discontinue all pain medications for condition under study,

except rescue medication permitted per protocol, for the duration of the study;

they must enter the required daily assessments during the PDEP for at least 5 of the 7 days prior to randomization.

### Exclusion Criteria:

Participants are excluded from the study if any of the following criteria apply:

they have a current drug-induced neuropathy, for example, due to some types of chemotherapy, or other types of peripheral neuropathy;

they have known hereditary motor, sensory or autonomic neuropathies.

Other Medical Conditions:

they have an eGFR of less than 70 ml/min/1.73m2 based on CKD-EPI formula at Visit 1 or Visit 2;

Have any clinically serious or unstable cardiovascular, musculoskeletal disorder, gastrointestinal, endocrinologic, hematologic, hepatic, metabolic, urologic, pulmonary, dermatologic, immunologic, or ophthalmologic disease within 3 months of Visit 3.

Prior/Concomitant Therapy: they have received any antibodies against nerve growth factor (NGF), or antibodies against EGFR, or EGFR tyrosine kinase inhibitors; have a history of allergic reactions to monoclonal antibodies, or clinically significant multiple or severe drug allergies, including but not limited to erythema multiforme major, linear immunoglobulin A dermatosis, toxic epidermal necrolysis, or exfoliative dermatitis; have a history or presence of uncontrolled asthma, eczema, significant atopy, significant hereditary angioedema or common variable immune deficiency, and

Reproductive: they are women who are pregnant or breastfeeding.

Participants are required to maintain similar levels of activity during the double-blind study period. Starting a new exercise program or new strenuous activity is not allowed.

### Pharmacokinetics:

Venous blood samples of approximately 4 mL will be collected for measurement of Antibody I concentrations. Samples will be used to evaluate the PK of Antibody I. Site personnel will record the date and time (24-hour clock time) of Antibody I administration (start and end of infusion), and the date and time (24-hour clock time) of each PK sample.

### Pharmacodynamics:

Venous blood samples of approximately 4 mL each will be collected for the measurement of epiregulin. Any remaining blood samples may be used to test other potential PD endpoints, including, but not limited to TGF-α.

### Immunogenicity Assessments:

Immunogenicity will be assessed by a validated assay designed to detect ADAs in the presence of Antibody I at a laboratory approved by the sponsor. Antibodies may be further characterized for their ability to neutralize the activity of Antibody I. At the visits and times specified, predose venous blood samples will be collected to determine antibody production against Antibody I. The actual date and time (24-hour clock time) of each sample collection will be recorded. If the immunogenicity sample at the last scheduled assessment or discontinuation visit is treatment-emergent (TE) anti-drug antibody (ADA) positive, additional samples may be taken until the signal returns to baseline (i.e., no longer TE-ADA positive) or for up to 1 year after last dose. To aid interpretation of these results, a predose blood sample for PK analysis will be collected at the same time points.

### Statistical Hypotheses:

A Bayesian critical success factor (CSF) is defined and used to evaluate whether Antibody I met its primary endpoint. The CSF will be evaluated for the primary efficacy endpoint, average pain intensity as measured by the NRS, using the methodology described herein and known to the skilled artisan, and will be calculated at the conclusion of the double-blind portion of each study. The CSF will have the general form of: probability (treatment effect<effect of interest)>probability threshold. The treatment effect will be defined as the Antibody I estimate-placebo estimate of the change from baseline at endpoint. The effect of interest is typically found through a literature search or clinical judgement. The probability threshold is generally set to have a desired level of confidence in the treatment effect or to have the desired operating characteristics under a range of plausible, assumed drug effect scenarios of truth, including a null effect. Additional hypotheses will include the comparison of the active intervention with placebo for the prespecified objectives and endpoints defined herein. The study may be conducted in a protocol wherein multiple studies are contemplated and placebo data may be shared as appropriate.

The decision criterion for the primary hypothesis is defined as at least 70% confidence that Antibody I is at least 0.4 units better than placebo on average pain intensity as measured by the NRS.

BPI-SF Continuous Efficacy Analysis: A Bayesian longitudinal mixed-effect model repeated measures (MMRM) analysis will be performed to evaluate the change from baseline to each postbaseline visit for the total pain interference scale. The analysis will be used to analyze the change from baseline to each postbaseline visit for: individual pain interference, total pain interference (sum of the 7 responses), and individual pain severity scales. The proportion of participants in each treatment group meeting prespecified binary efficacy outcomes will be estimated for each postbaseline time point and will be used to compare treatment groups. The estimates will be provided from fitting a Bayesian longitudinal model that includes all postbaseline observations. The prespecified binary efficacy outcomes include the proportion of participants with a reduction >30%, 50% and 70% from baseline as measured by the BPI-SF individual severity scores, and with a reduction >30%, 50% and 70% from baseline as measured by the BPI-SF total interference score.

### Sample Size Determination:

Approximately 125 participants will be randomized in a 2:1 ratio to Antibody I and placebo, respectively. It is expected that approximately 107 participants will complete the double-blind treatment period of the study. This sample size will provide greater than 80% power to demonstrate that the active treatment arm has a >0.7 posterior probability of being better than placebo by at least 0.4 units on average pain intensity as measured by the NRS.

The assumption for the power calculation is that the mean reduction in average pain intensity, as measured by the NRS, are approximately 1.58 units and 2.58 units at endpoint, for placebo and Antibody I, respectively, with a common standard deviation of 2. If there is no treatment difference between placebo and Antibody I, the probability of passing the efficacy criterion specified above (i.e., false positive) is approximately 0.06. The simulation for the power calculation and sample size determination was carried out in FACTS Version 6.0.

### Populations for Analyses:

The pharmacokinetic population includes for example all randomized participants who received a full dose of Antibody I at Visit 3 and have at least 1 evaluable PK sample collected prior to dosing at or after Visit 4.

### Example 9: Randomized, Placebo-Controlled, Phase 2 Clinical Trial to Evaluate Antibody I for the Treatment of Chronic Low Back Pain

The purpose of this study is to provide human clinical evidence for Antibody I efficacy in relieving pain due to Chronic Low Back Pain (CLBP). Data will be collected to assess the safety, and tolerability of Antibody I in this study population.

Pharmacokinetic (PK) properties, pharmacodynamic (PD) effects and immunogenicity profile will also be explored. The totality of data from this proof-of-concept study will assess the benefits and risks associated with Antibody I and inform the clinical development of Antibody I.

### Overall Design:

This is a 26-week, Phase 2, randomized, double-blind, placebo-controlled study that will compare Antibody I versus placebo in participants with CLBP. This is a randomized, investigator- and participant-blind, placebo controlled, Phase 2 clinical trial. Approximately 150 participants will be randomly assigned to study intervention (100 Antibody I and 50 placebo). This 26-week study includes an 8-week double-blind treatment period and an 18-week follow-up period. This study design is shown in FIG. 2. Note that methods and specifications and procedures may be in common with Examples 7, 8 and 9, and the skilled artisan will recognize they need not be repeated in each example, and similarly, additional routine testing for health and adverse events, as is typical for clinical studies, is understood to be part of the design and execution of studies of Example 7, 8, and 9.

The study intervention will be administered via a slow intravenous (IV) infusion over approximately 1 hour by blinded site personnel. The infusion rate may be reduced as deemed necessary if an infusion reaction is observed. The dose is a 750-mg starting dose followed by 500 mg every 2 weeks IV for a total of 4 doses. Dose formulation is a lyophilized powder reconstituted with sterile water 0.9% Sodium chloride solution. Participants will receive an IV infusion every 2 weeks for a total of 4 infusions. Participants will be monitored for at least 4 hours after completion of each infusion.

At patient visits, all post-treatment sample collection and safety monitoring are completed, and participants are instructed to continue with study restrictions and Numeric Rating Scale (NRS) diary entries before their visit discharge. Efficacy data will be collected up to 6 weeks after the last dose, based on the long PK half-life and potential sustained target engagement of Antibody I. Safety, pharmacokinetic (PK), pharmacodynamic (PD) and immunogenicity samples will be collected up to 20 weeks after the last administration of intervention to characterize the safety and clinical immunogenicity profile. A participant is considered to have completed this study if he or she has completed all required phases of the study including the last scheduled procedure.

### Objectives and Endpoints:

Efficacy Outcome Measures: Primary outcome measures include: Change from Baseline for Average Pain Intensity as Measured by the Numeric Rating Scale (NRS), Change from Baseline for Average Pain Intensity as Measured by the NRS [Time Frame: Baseline, up to Week 8], Secondary outcome measures include: Change from Baseline on the Roland Morris Disability Questionnaire (RMDQ), Change from Baseline on the RMDQ [Time Frame: Baseline, up to Week 8], Change from Baseline for Overall Improvement as Measured by Patient's Global Impression of Change, Change from Baseline for Overall Improvement as Measured by Patient's Global Impression of Change [Time Frame: Baseline, up to Week 8], Change from Baseline for Worst Pain Intensity as Measured by NRS, Change from Baseline for Worst Pain Intensity as Measured by NRS [Time Frame: Baseline, up to Week 8], Change from Baseline on the Visual Analog Scale (VAS) for Pain, Change from Baseline on the VAS for Pain [Time Frame: Baseline, up to Week 8], Change from Baseline on the Sleep Scale from the Medical Outcomes Study (MOS Sleep Scale), Change from Baseline on the Sleep Scale from the MOS Sleep Scale [Time Frame: Baseline, up to Week 8], Total Amount of Rescue Medication Total Amount of Rescue Medication [Time Frame: Baseline up to Week 8], Change from Baseline on the EuroQol-5D 5 Level Questionnaire (EQ-5D-5L), Change from Baseline on the EQ-5D-5L.

### Overview of Assessments:

| Core Assessment | Measure |
|---|---|
| Pain intensity | Visual Analog Scale (VAS) for pain |
| Pain intensity | Numeric Rating Scale (NRS) for pain |
| Participant ratings | Patient Global Impression of Change (PGI) |
| of overall improvement | |
| Emotional functioning | EuroQol-5D 5 level questionnaire (EQ-5D-5L) |
| Quality of sleep | Medical Outcomes Study (MOS) Sleep Scale |
| Physical functioning | Change from baseline to endpoint for the |
| Efficacy of each study | Roland-Morris Disability Questionnaire |
| intervention | (RMDQ) |
| versus placebo | Proportion of participants with reduction |
| | from baseline of at least 3.5 points on |
| | RMDQ total score across all time points |
| | Proportion of participants with reduction |
| | from baseline greater than 30%, 50%, |
| | and 70% on the RMDQ across all time |
| | points |

### Efficacy Assessments:

| Objective | Endpoint Measure |
|---|---|
| Pain Intensity: | Mean change from baseline assessment to |
| Efficacy of Antibody | endpoint for average pain intensity as |
| I versus placebo | measured by numeric rating scale (NRS) |
| Overall Improvement: | Mean change from baseline assessment to |
| Efficacy of Antibody | endpoint for overall improvement as |
| I versus placebo | measured by Patient's Global Impression of |
| | Change |
| Efficacy of Antibody | Mean change from baseline |
| I versus placebo | assessment to endpoint for worst |
| | pain intensity as measured by NRS |
| | Proportion of patients with a pain |
| | reduction from baseline greater than |
| | 30%, 50%, and 70% as measured by |
| | the average and worst pain |
| | responses on the NRS |
| | Proportion of patients with 2-point |
| | reduction from baseline as |
| | measured by the average and worst |
| | pain responses on the NRS |
| | Time to treatment response with a |
| | 30%, 50%, and 70% reduction from |
| | baseline as measured by the average |
| | and worst pain responses on the |
| | NRS |
| | Time to treatment response with 2- |
| | point reduction from baseline as |
| | measured by the average and worst |
| | pain |
| | Mean change from baseline |
| | assessment to endpoint on the visual |
| | analog scale (VAS) for pain |
| | responses on the NRS |
| | Proportion of patients with pain |
| | reduction from baseline greater than |
| | 30%, 50%, and 70% as measured by |
| | VAS |
| | Mean change from baseline |
| | assessment to endpoint on the Sleep |
| | Scale from the Medical Outcomes |
| | Study (MOS Sleep Scale) |
| | Proportion of patients with |
| | reduction from baseline greater than |
| | 30%, 50%, and 70% on the physical |
| | functioning measures as described |
| | for RMDQ, and |
| | Summary of frequency, timing, and |
| | amount of rescue medication used |
| | during the treatment phase. |
| Emotional Functioning: | Mean change from baseline assessment to |
| Efficacy of each | endpoint for emotional functioning as |
| study intervention | measured by the EuroQol-5D 5 level |
| versus placebo on | questionnaire (EQ-5D-5L) |
| patient-reported | |
| clinical outcomes | |

A core set of assessments and domains are used when characterizing chronic pain, which are: pain, physical functioning, emotional functioning, participant ratings of overall improvement, adverse events (Aes), and participant disposition. The NRS is selected for the primary endpoint based on its demonstrated ability to detect changes in pain and its common use across the disease states under study.

Visual Analog Scale (VAS): The VAS for pain will be used at screening and at each clinic visit. This is a graphic, single-item scale where participants are asked to describe their pain intensity over the past week, in the area under study, on a scale of 0 to 100:0=no pain, and 100=worst imaginable pain. Participants complete the VAS by placing a line perpendicular to the VAS line at a point that describes their pain intensity. Numeric Rating Scale (NRS): The NRS will be used during the preliminary data entry period (PDEP) and daily throughout the study to describe pain severity. This is a numeric, single-item scale where participants are asked to describe their average and worst pain over the past 24 hours, on a scale of 0 to 10:0=no pain, and 10=pain as bad as you can imagine. Participants complete the NRS daily using a take-home device. Participant compliance is reviewed at each clinic visit. The NRS worst pain will be collected daily. The scores for the other secondary measures will be collected at each visit as specified in the visit schedule. The primary outcome measure is the mean change from baseline to endpoint for average pain intensity as assessed by the NRS item ‘Please rate your pain by selecting the one number [0-10] that describes your AVERAGE level of [area under study] pain during the past 24 hours.’ This measure was selected based on its demonstrated ability to detect changes in pain and its common use across disease states. A secondary measure is the mean change from baseline to endpoint for worst pain intensity as measured by the NRS item ‘Please rate your pain by selecting the one number [0-10] that describes your WORST level of [area under study] pain during the past 24 hours.’ The NRS value of average pain and worst pain over the past 24 hours will be collected daily for each participant. For the statistical analyses, the average NRS value of the average and worst pain over the past 24 hours will be calculated for both weekly intervals and biweekly intervals. The average of the weekly intervals for the NRS will result in 8 postbaseline observations, and the average of the biweekly intervals will result in 4 postbaseline observations for each participant if a participant completes the placebo-controlled portion of the study. The average of the weekly intervals for the NRS will be used in the primary efficacy analysis and other analyses described below, unless otherwise specified in the analysis plan. A participant must have 50% or greater of the daily NRS values during the prespecified time interval to calculate the average NRS value; otherwise, the average NRS value for that visit will be considered missing.

Participant Ratings on Overall Improvement: The Patient Global Impression of Change (PGI) is used across disease states. It captures the participant's perspective of treatment apart from sub-aspects of the general improvement. This is a numeric scale from 1 to 7:1=very much better, and 7=very much worse. The participant is asked to ‘Mark the box that best describes how your pain symptoms are now, compared to how they were before you started taking this medicine.’

Emotional Functioning Assessments: The EQ-5D-5L health status questionnaire is used across disease states. The EQ-5D-5L is one of the most popular patient-completed instruments to address quality of life (Buchholz I, Janssen M F, Kohlmann T, Feng Y S. A systematic review of studies comparing the measurement properties of the three-level and five-level versions of the EQ-5D. PharmacoEconomics. 2018; 36(6):645-661.). It is a descriptive system that includes 5 dimensions: mobility, self-care, usual activities, pain/discomfort, and anxiety/depression. The participant is asked to ‘check the ONE box that best describes your health TODAY,’ choosing from 5 options provided under each dimension. The scores on the 5 dimensions can be presented as a health profile or converted to a single summary index number. The EQ-5D-5L also includes the EQ VAS, which records the participant's self-rated health on a vertical VAS of 0 to 100:0=the worst health you can imagine, and 100=the best health you can imagine. The instrument used in its EQ-5D-5L version is a short, reliable, validated, easy-to-complete scale with excellent test-retest reliability to address quality of life in relation to pain due to several diseases.

Sleep Quality: Sleep disturbance is an important issue in pain research. Among various available instruments, the Medical Outcome Study (MOS) Sleep Scale provides a unique, psychometrically validated score for sleep disturbance. This scale consists of 12 questions addressing the past week. Participants report how often each sleep symptom or problem is present on a 6-point categorical scale ranging from ‘all of the time’ to ‘none of the time.’ Questions about time to fall asleep and quantity of sleep are reported as the average number of hours slept each night.

Safety Assessments: Planned time points for safety assessments are determined according to typical practices, and include physical examination, vital sign and body weight measurements, 12-lead ECGs, clinical laboratory tests, hepatic safety monitoring, C-SSRS, and spontaneously reported Aes.

Roland Morris Disability Questionnaire: The Roland Morris Disability Questionnaire (RMDQ) is a simple, sensitive, and reliable method to measure disability in patients with back pain. The RMDQ consists of 24 statements relating to the person's perceptions of back pain and associated disability based on: physical ability/activity, sleep/rest, psychosocial, household management, eating, and pain frequency. Participants are asked if they feel the statement is descriptive of their own circumstance on that day. The total score is obtained by counting the number of “Yes” responses, ranging from: 0=no disability to 24=maximal disability.

PainDETECT: The painDETECT questionnaire consists of 7 questions on the quality of neuropathic pain symptoms. It was originally proposed to capture the neuropathic pain phenotype in patients with low back pain (Freynhagen 2006). It is easily answered by the participant and does not require a physical examination. A score of ≥19 indicates that pain is likely phenotypically neuropathic (>90%).

### Statistical Analyses:

This table defines the stratification factor for chronic low back pain.

| Neuropathic pain | painDETECT score |
|---|---|
| Positive presence | ≥19 |
| Unclear or negative | <19 |

Any additional stratification factors may be defined in the ISA.

### Endpoint Analysis—RMDQ

Continuous Efficacy Analysis: A Bayesian longitudinal mixed-effect model repeated measures (MMRM) analysis will be performed to evaluate the change from baseline to each post baseline visit for the RMDQ score. The same placebo borrowing strategy will be implemented as described in the master protocol; however, no treatment effect borrowing from other DSAs will be performed. An additional Bayesian MMRM analysis will also be conducted using data only from the respective ISA, which does not utilize any borrowing of placebo information. The analysis will be used to analyze the change from baseline to each post baseline visit for the RMDQ score. (See Freynhagen R, Baron R, Gockel U, Tölle T R. painDETECT: a new screening questionnaire to identify neuropathic components in patients with back pain. Curr Med Res Opin. 2006; 22(10):1911-1920.)

This table describes information included in the model.

| | |
|---|---|
| Categorical effects | treatment |
| | visit, and |
| | the interaction of treatment and visit |
| Continuous covariates | baseline RMDQ score, and |
| | the interaction of baseline RMDQ score |
| | by visit |

### Categorical Efficacy Analysis:

The proportion of participants in each treatment group meeting prespecified binary efficacy outcomes will be estimated for each post baseline time point and will be used to compare treatment groups. The estimates will be provided from fitting a Bayesian longitudinal model that includes all post baseline observations.

The prespecified binary efficacy outcomes include the proportion of participants within an ISA:

with a reduction greater than 30%, 50%, and 70% from baseline as measured by the RMDQ score, and

with at least a 3.5 point reduction from baseline in the RMDQ score.

The model will include the categorical and continuous covariates described in the continuous efficacy analysis model above, except the interaction of baseline and visit will not be used. A cumulative distribution function of percent change from baseline to endpoint for the RMDQ scores will be provided for each treatment group.

### Schedule of Activities (SoA):

This schedule of activities shows certain visits and procedures for a study of Antibody I. “Master protocol” refers a protocol setup to guide several potential studies, in a given disease state or multiple disease states, and intervention specific addendum refers to the part of the protocol specifically related to a given intervention under study.

| | Screening^{a} | | | | | | | | |
|---|---|---|---|---|---|---|---|---|---|
| | | Pre- | | | | | | | |
| | Screening | Randomization | Double-Blind Treatment Period | | | | | | |
| | Visit for | Screening | Randomization | | | | | Early | |
| | Master | Visit | to ISA | | | | | Discontinuation | Notes |
| | Visit Number | | | | | | | | |
| | | | | | | | | | Visits after V7 |
| | | | | | | | | | may be specified in the |
| | V1^{b} | V2^{c} | V3 | V4 | V5 | V6 | V7 | ED | ISA. |
| | Study Week | | | | | | | | |
| | | −10 to −7 | | | | | | | |
| H0P-MC-CPMP | | days for | | | | | | | |
| Master | Up to −6 | most | | | | | | | |
| Protocol | months | participants | 0 | 2 | 4 | 6 | 8 | | |
| VAS for pain | X | X | X | X | X | X | X | X | |

Scales, Questionnaires, and Outcome Measures

| | | | | | | | | | |
|---|---|---|---|---|---|---|---|---|---|
| NRS for pain | | X | X | X | X | X | X | X | Collected daily with a |
| | | | | | | | | | take-home device. |
| | | | | | | | | | Compliance reviewed at |
| | | | | | | | | | each clinic visit. |
| Rescue | | X | X | X | X | X | X | X | Total quantity collected |
| medication usage | | | | | | | | | daily with a take-home |
| reporting | | | | | | | | | device. Compliance |
| | | | | | | | | | reviewed at each clinic |
| | | | | | | | | | visit. |
| Pain | X | | | | | | | | |
| catastrophizing | | | | | | | | | |
| scale | | | | | | | | | |
| PGI | | | | X | X | X | X | X | |
| EQ-5D-5L | | | X | X | X | X | X | X | |
| MOS Sleep Scale | | | X | X | X | X | X | X | |

| | Screening^{a} | | | | | | | | |
|---|---|---|---|---|---|---|---|---|---|
| | | Pre- | | | | | | | |
| | | Randomization | | | | | | | |
| | Screening | Screening | Double-Blind Treatment Period | | | | | Early | |
| | Visit | Visit | Randomization | | | | | Discontinuation | Notes |
| | Visit Number | | | | | | | | |
| | V1^{b} | V2^{c} | V3 | V4 | V5 | V6 | V7 | ED | |
| | Study Week | | | | | | | | |
| H0P-MC- | Up to −6 | | | | | | | | |
| BP02 DSA | months | −7 to −10 days | 0 | 2 | 4 | 6 | 8 | | |
| Procedure | | | | | | | | | |
| Confirm DSA | X | X | | | | | | | |
| inclusion and | | | | | | | | | |
| exclusion | | | | | | | | | |
| criteria | | | | | | | | | |
| X-ray | X | | | | | | | | Lumbar spinal |
| | | | | | | | | | anterior/posterior, |
| | | | | | | | | | lateral x-rays with |
| | | | | | | | | | flexion and |
| | | | | | | | | | extension views |
| Roland Morris | | | X | X | X | X | X | X | |
| Disability | | | | | | | | | |
| Questionnaire | | | | | | | | | |
| painDETECT | X | | | | | | | | |

Abbreviations: DSA = disease-state addendum; ED = early discontinuation; V = visit.
^{a}Screening assessments may be conducted at other time points prior to randomization if they reduce participant burden.
^{b}The site determines the half-life of each pain medication the participant is currently taking in order to schedule Visit 2. Visit 2 can be scheduled no earlier than 7 days prior to randomization at Visit 3 due to the required 7 -day PDEP
^{c}The 5 half-life washout period for pain medications must come before the PDEP, resulting in a minimum of 10 days for most participants.

Study Population: Male and female participants are eligible for inclusion in the study if they have a history of daily pain based on patient report or medical history.

Informed Consent: they are capable of providing informed consent, which includes compliance with the requirements and restrictions listed in the informed consent form (ICF) and in the protocol; they are reliable, willing, and able to participate in all required protocol procedures for the duration of the study; they are willing to maintain a consistent regimen of any ongoing nonpharmacologic pain-relieving therapies (for example, physical therapy) and will not start any new nonpharmacologic pain-relieving therapies during study participation; they are willing to discontinue all pain medications for condition under study, except rescue medication permitted per protocol, for the duration of the study; they must enter the required daily assessments during the PDEP for at least 5 of the 7 days prior to randomization.

### Inclusion Criteria:

Participants are eligible to be included in the study only if all the following criteria apply: are 18 years or older in age at the time of signing the informed consent;

Have a visual analog scale (VAS) pain value ≥40 and <95 during screening.

Have a history of daily pain for at least 12 weeks based on participant report or medical history.

Have a value of ≤30 on the pain catastrophizing scale.

Have a body mass index <40 kilograms per meter squared (kg/m²) (inclusive).

Are willing to maintain a consistent regimen of any ongoing nonpharmacologic pain-relieving therapies (for example, physical therapy) and will not start any new nonpharmacologic pain-relieving therapies during study participation.

Are willing to discontinue all pain medications for condition under study, except rescue medication permitted per protocol, for the duration of the study.

Have a history of low back pain for at least 3 months located between the 12th thoracic vertebra and the lower gluteal folds, with or without radiation.

Have a history of low back pain as classified by the Quebec Task Force Category 1 through 3.

Have stable glycemic control as indicated by a glycated hemoglobin (HbA1c) less than or equal to 10 at time of screening.

Have an estimated glomerular filtration rate (eGFR) of less than 70 ml/min/1.73 m² during screening.

Are men, or women able to abide by reproductive and contraceptive requirements.

### Exclusion Criteria:

Participants are excluded from the study if any of the following criteria apply:
Have any clinically serious or unstable cardiovascular, musculoskeletal disorder, gastrointestinal (GI), endocrinologic, hematologic, hepatic, metabolic, urologic, pulmonary, dermatologic, immunologic, or ophthalmologic disease within 3 months of baseline.
Have received any antibodies against nerve growth factor (NGF), antibodies against EGFR, or EGFR tyrosine kinase inhibitors (TKI).
Have a history of allergic reactions to monoclonal antibodies, or clinically significant multiple or severe drug allergies, including but not limited to erythema multiforme major, linear immunoglobulin A dermatosis, toxic epidermal necrolysis, or exfoliative dermatitis.
Have a history or presence of uncontrolled asthma, eczema, significant atopy, significant hereditary angio-edema, or common variable immune deficiency.
Have used a therapeutic injection (botulinum toxin or corticosteroids) in the 3 months prior to starting the washout period;
Have history of or current osteoporotic compression fracture; have had a recent major trauma (within 6 months of V3);
Have had surgical intervention for the treatment of low back pain in the past 6 months.
Reproductive: they are women who are pregnant or breastfeeding.
Prior/Concomitant Therapy: they have received any antibodies against nerve growth factor (NGF), or antibodies against EGFR, or EGFR tyrosine kinase inhibitors; have a history of allergic reactions to monoclonal antibodies, or clinically significant multiple or severe drug allergies, including but not limited to erythema multiforme major, linear immunoglobulin A dermatosis, toxic epidermal necrolysis, or exfoliative dermatitis; have a history or presence of uncontrolled asthma, eczema, significant atopy, significant hereditary angioedema or common variable immune deficiency, and

### Study Assessments and Procedures:

Pharmacokinetics: Venous blood samples of approximately 4 mL will be collected for measurement of Antibody I concentrations. Samples will be used to evaluate the PK of Antibody I. Site personnel will record the date and time (24-hour clock time) of Antibody I administration (start and end of infusion), and the date and time (24-hour clock time) of each PK sample.

Pharmacodynamics: Venous blood samples of approximately 4 mL each will be collected for the measurement of epiregulin. Any remaining blood samples may be used to test other potential PD endpoints, including, but not limited to TGF-α.

Immunogenicity Assessments: Immunogenicity will be assessed by a validated assay designed to detect ADAs in the presence of Antibody I at a laboratory approved by the sponsor. Antibodies may be further characterized for their ability to neutralize the activity of Antibody I. At the visits and times specified, predose venous blood samples will be collected to determine antibody production against Antibody I. The actual date and time (24-hour clock time) of each sample collection will be recorded. If the immunogenicity sample at the last scheduled assessment or discontinuation visit is treatment-emergent (TE) anti-drug antibody (ADA) positive, additional samples may be taken until the signal returns to baseline (i.e., no longer TE-ADA positive) or for up to 1 year after last dose. To aid interpretation of these results, a predose blood sample for PK analysis will be collected at the same time points.

### Statistical Hypotheses:

A Bayesian critical success factor (CSF) is defined and used to evaluate whether Antibody I met its primary endpoint. The CSF will be evaluated for the primary efficacy endpoint, average pain intensity as measured by the NRS, using the methodology described herein and known to the skilled artisan, and will be calculated at the conclusion of the double-blind portion of each study. The CSF will have the general form of: probability (treatment effect<effect of interest)>probability threshold. The treatment effect will be defined as the Antibody I estimate-placebo estimate of the change from baseline at endpoint. The effect of interest is typically found through a literature search or clinical judgement. The probability threshold is generally set to have a desired level of confidence in the treatment effect or to have the desired operating characteristics under a range of plausible, assumed drug effect scenarios of truth, including a null effect. Additional hypotheses will include the comparison of the active intervention with placebo for the prespecified objectives and endpoints defined herein. The study may be conducted in a protocol wherein multiple studies are contemplated, and placebo data may be shared as appropriate.

The decision criterion for the primary hypothesis is defined as being at least 70% confident that Antibody I is at least 0.55 units better than placebo on average pain intensity as measured by the NRS. The key secondary null hypothesis is that there is no difference between Antibody I and placebo on the key secondary endpoint, the mean change from baseline to endpoint for the relevant pain score. The decision criterion for the key secondary hypothesis is defined as being at least 70% confident that Antibody I is at least 0.35 units better than placebo on the relevant pain score.

Populations for Analyses: The pharmacokinetic population includes for example all randomized participants who received a full dose of Antibody I at Visit 3 and have at least 1 evaluable PK sample collected prior to dosing at or after Visit 4.

Listing Amino Acid and Nucleic Acid Sequences

Heavy Chain CDRs
SEQ ID NO: 1 GYTFTDAYIN
SEQ ID NO: 2 WIWPGPVITYYNPKFKG
SEQ ID NO: 3 REVLSPFAY
Light Chain CDRs
SEQ ID NO: 4 RSSQSIVHSTGNTYLE
SEQ ID NO: 5 KVSNRFS
SEQ ID NO: 6 FHGTHVPYT
Heavy Chain Variable Regions
SEQ ID NO: 7 (Antibody I and Antibody II)
QVQLVQSGAEVKKPGSSVKVSCKASGYTFTDAYINWVRQAPGQGLEWMGWIW
PGPVITYYNPKFKGRVTITADKSTSTAYMELSSLRSEDTAVYYCARREVLSPFAY
WGQGTTVTVSS
SEQ ID NO: 8 (Antibody III)
QVQLQQSGPELVKPGASVKISCKASGYTFTDAYINWVKQRPGQGLEWIGWIWPG
PVITYYNPKFKGKATLTVDKSSSTAYMLLSSLTSEDSAFYFCARREVLSPFAWG
QGTLVTVSA
Light Chain Variable Regions
SEQ ID NO: 9 (Antibody I)
DIVMTQSPDSLAVSLGERATINCRSSQSIVHSTGNTYLEWYQQKPGQPPKWYKV
SNRFSGVPDRFSGSGSGTDFTLTISSLQAEDVAVYYCFHGTHVPYTFGGGTKVEIK
SEQ ID NO: 10 (Antibody II)
DIQMTQSPSSLSASVGDRVTITCRSSQSIVHSTGNTYLEWYQQKPGKAPKLLIYKV
SNRFSGVPSRFSGSGSGTDFTLTISSLQPEDFATYYCFHGTHVPYTFGGGTKVEIK
SEQ ID NO: 11 (Antibody III)
DVLMTQTPLSLPVSLGDQASISCRSSQSIVHSTGNTYLEWYLQKPGQSPKLLIYKV
SNRFSGVPDRFSGSGSGTDFTLKISRVEAEDLGVYYCFHGTHVPYTFGGGTKLEIK
Complete Heavy Chain
SEQ ID NO: 12 (Antibody I and Antibody II)
QVQLVQSGAEVKKPGSSVKVSCKASGYTFTDAYINWVRQAPGQGLEWMGWIW
PGPVITYYNPKFKGRVTITADKSTSTAYMELSSLRSEDTAVYYCARREVLSPFAY
WGQGTTVTVSSASTKGPSVFPLAPCSRSTSESTAALGCLVKDYFPEPVTVSWNSG
ALTSGVHTFPAVLQSSGLYSLSSVVTVPSSSLGTKTYTCNVDHKPSNTKVDKRVE
SKYGPPCPPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYT
QKSLSLSLG
Complete Light Chains
SEQ ID NO: 13 (Antibody I)
DIVMTQSPDSLAVSLGERATINCRSSQSIVHSTGNTYLEWYQQKPGQPPKWYKV
SNRFSGVPDRFSGSGSGTDFTLTISSLQAEDVAVYYCFHGTHVPYTFGGGTKVEIK
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
SEQ ID NO: 14 (Antibody II)
DIQMTQSPSSLSASVGDRVTITCRSSQSIVHSTGNTYLEWYQQKPGKAPKLLIYKV
SNRFSGVPSRFSGSGSGTDFTLTISSLQPEDFATYYCFHGTHVPYTFGGGTKVEIKR
TVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
Nucleotide Sequences
Heavy Chain Variable Region
SEQ ID NO: 15
CAGGTGCAGCTGGTGCAGTCTGGGGCTGAGGTGAAGAAGCCTGGGTCCTCAG
TGAAGGTTTCCTGCAAGGCATCTGGCTACACCTTCACTGACGCGTATATAAAC
TGGGTGCGACAGGCCCCTGGACAAGGGCTTGAGTGGATGGGATGGATTTGGC
CTGGACCCGTTATTACTTACTACAATCCGAAGTTCAAGGGCAGAGTCACCATT
ACCGCGGACAAATCCACGAGCACAGCCTACATGGAGCTGAGCAGCCTGAGAT
CTGAGGACACGGCCGTGTATTACTGTGCGAGAAGGGAAGTACTATCCCCGTT
TGCTTACTGGGGCCAAGGAACCACGGTCACCGTCTCCTCA
Nucleotide Sequences
Light Chain Variable Regions
SEQ ID NO: 16
GACATCGTGATGACCCAGTCTCCAGACTCCCTGGCTGTGTCTCTGGGCGAGAG
GGCCACCATCAACTGCAGATCTAGTCAGAGCATTGTACATAGTACTGGAAAC
ACCTATTTAGAATGGTACCAGCAGAAACCAGGACAGCCTCCTAAGCTGCTCA
TTTACAAAGTTTCCAACCGATTTTCTGGGGTCCCTGACCGATTCAGTGGCAGC
GGGTCTGGGACAGATTTCACTCTCACCATCAGCAGCCTGCAGGCTGAAGATG
TGGCAGTTTATTACTGTTTTCACGGCACTCATGTTCCGTACACGTTCGGCGGA
GGGACCAAGGTGGAGATCAAA
SEQ ID NO: 17
GACATCCAGATGACCCAGTCTCCATCCTCTCTGTCTGCATCTGTAGGAGACAG
AGTCACCATCACTTGCAGATCTAGTCAGAGCATTGTACATAGTACTGGAAAC
ACCTATTTAGAATGGTATCAGCAGAAACCAGGGAAAGCCCCTAAGCTCCTGA
TCTATAAAGTTTCCAACCGATTTTCTGGGGTCCCATCAAGGTTCAGTGGCAGT
GGATCTGGGACAGATTTCACTCTCACCATCAGCAGTCTGCAACCTGAAGATTT
TGCAACTTACTACTGTTTTCACGGCACTCATGTTCCGTACACGTTCGGCGGAG
GGACCAAGGTGGAGATCAAA
Mature Human TGF alpha
SEQ ID NO: 18
VVSHFNDCPDSHTQFCFHGTCRFLVQEDKPACVCHSGYVGARCEHADLLA
Mature Mouse (Mus musculus) TGF alpha
SEQ ID NO: 19
VVSHFNKCPDSHTQYCFHGTCRFLVQEEKPACVCHSGYVGVRCEHADLLA
Mature Rat (Rattus noryegicus) TGF alpha
SEQ ID NO: 20
VVSHFNKCPDSHTQYCFHGTCRFLVQEEKPACVCHSGYVGVRCEHADLLA
Mature Cyno (Macaca fascicularis) TGF alpha
SEQ ID NO: 21
VVSHFNDCPDSHTQFCFHGTCRFLVQEDKPACVCHSGYVGARCEHADLLA
Mature Human Epiregulin - addition of N-terminal methionine
SEQ ID NO: 22
MVSITKCSSDMNGYCLHGQCIYLVDMSQNYCRCEVGYTGVRCEEIFFL
Mature Mouse (Mus musculus) Epiregulin - addition of N-terminal methionine
SEQ ID NO: 23
MVQITKCSSDMDGYCLHGQCIYLVDMREKFCRCEVGYTGLRCEEIFFL
Mature Cyno (Macaca fascicularis) Epiregulin
SEQ ID NO: 24
VSITKCNSDMNGYCLHGQCIYLVDMSQNYCRCEVGYTGVRCEHFYL
Mature Human Epigen
SEQ ID NO: 25
AVTVTPPITAQQADNIEGPIALKFSHLCLEDHNSYCINGACAFHHELEKAICRCFT
GYTGERCEHLTLTSYA
Mature Mouse (Mus musculus) Epigen
SEQ ID NO: 26
LKFSHPCLEDHNSYCINGACAFHHELKQAICRCFTGYTGQRCEHLTLTSYA
Mature Human EGF - addition of N-terminal methionine
SEQ ID NO: 27
MNSDSECPLSHDGYCLHDGVCMYIEALDKYACNCVVGYIGERCQYRDLKWWE
LR
Mature Human HBEGF
SEQ ID NO: 28
DLQEADLDLLRVTLSSKPQALATPNKEEHGKRKKKGKGLGKKRDPCLRKYKDF
CIHGECKYVKELRAPSCICHPGYHGERCHGLSL
Mature Human Betacellulin
SEQ ID NO: 29
DGNSTRSPETNGLLCGDPEENCAATTTQSKRKGHFSRCPKQYKHYCIKGRCRFV
VAEQTPSCVCDEGYIGARCERVDLFY
Mature Human Amphiregulin
SEQ ID NO: 30
SVRVEQVVKPPQNKTESENTSDKPKRKKKGGKNGKNRRNRKKKNPCNAEFQNF
CIHGECKYIEHLEAVTCKCQQEYFGERCGEKSMKTHSMIDSSLSK
Complete Heavy Chain Antibody III - Mouse Antibody
SEQ ID NO: 31
QVQLQQSGPELVKPGASVKISCKASGYTFTDAYINWVKQRPGQGLEWIGWIWPG
PVITYYNPKFKGKATLTVDKSSSTAYMLLSSLTSEDSAFYFCARREVLSPFAYWG
QGTLVTVSAAKTTPPSVYPLAPGSAAQTNSMVTLGCLVKGYFPEPVTVTWNSGS
LSSGVHTFPAVLQSDLYTLSSSVTVPSSTWPSETVTCNVAHPASSTKVDKKIVPRD
CGCKPCICTVPEVSSVFIFPPKPKDVLTITLTPKVTCVVVDISKDDPEVQFSWFVDD
VEVHTAQTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQPAENY
KNTQPIMDTDGSYFVYSKLNVQKSNWEAGNTFTCSVLHEGLHNHHTEKSLSHSP
GK
Complete Light Chain Antibody III - Mouse Antibody
SEQ ID NO: 32
DVLMTQTPLSLPVSLGDQASISCRSSQSIVHSTGNTYLEWYLQKPGQSPKLLIYKV
SNRFSGVPDRFSGSGSGTDFTLKISRVEAEDLGVYYCFHGTHVPYTFGGGTKLEIK
RADAAPTVSIFPPSSEQLTSGGASVVCFLNNFYPKDINVKWKIDGSERQNGVLNS
WTDQDSKDSTYSMSSTLTLTKDEYERHNSYTCEATHKTSTSPIVKSFNRNEC
Mature Human Epiregulin
SEQ ID NO: 33
VSITKCSSDMNGYCLHGQCIYLVDMSQNYCRCEVGYTGVRCEHFFL

We claim:

1. A method of treating chronic pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

2. A method according to claim 1 of treating chronic osteoarthritis pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

3. A method according to claim 1 of treating chronic diabetic peripheral neuropathy pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

4. A method according to claim 1 of treating chronic low back pain in a subject in need thereof, comprising administering to the subject a therapeutically effective amount of an antibody comprising a light chain and a heavy chain, wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is SEQ ID NO:4, LCDR2 is SEQ ID NO:5, LCDR3 is SEQ ID NO:6, HCDR1 is SEQ ID NO:1, HCDR2 is SEQ ID NO:2, and HCDR3 is SEQ ID NO:3.

5. The method according to claim 1, wherein the amino acid sequence of the LCVR is SEQ ID NO:9 or SEQ ID NO:10.

6. The method according to claim 1, wherein the amino acid sequence of the HCVR is SEQ ID NO:7.

7. The method according to claim 1, wherein the amino acid sequence of the LCVR is SEQ ID NO:9 and the amino acid sequence of the HCVR is SEQ ID NO:7.

8. The method according to claim 1, wherein the amino acid sequence of the light chain is SEQ ID NO:13 or SEQ ID NO:14.

9. The method according to claim 1, wherein the amino acid sequence of the heavy chain is SEQ ID NO:12.

10. The method according to claim 1, wherein the antibody comprises two light chains wherein the amino acid sequence of each light chain is SEQ ID NO:13, and two heavy chains wherein the amino acid sequence of each heavy chain is SEQ ID NO:12.

11. The method according to claim 1, wherein the antibody comprises two light chains wherein the amino acid sequence of each light chain is SEQ ID NO:14, and two heavy chains wherein the amino acid sequence of each heavy chain is SEQ ID NO:12.

12. The method according to claim 1, wherein the dose of antibody is a 750 mg starting dose, followed by a 500 mg dose every 2 weeks, for as long as the patient needs treatment for pain.

13. The method according to claim 1 wherein the chronic pain is refractory to two or more prior monotherapy and/or dual therapy treatment regimens.


SEQUENCE LISTING (33 sequences)

SEQ ID NO: 1 - protein, 10 aa, Artificial Sequence
  synthetic construct
GYTFTDAYIN

SEQ ID NO: 2 - protein, 17 aa, Artificial Sequence
  Synthetic Construct
WIWPGPVITYYNPKFKG

SEQ ID NO: 3 - protein, 9 aa, Artificial Sequence
  Synthetic Construct
REVLSPFAY

SEQ ID NO: 4 - protein, 16 aa, Artificial Sequence
  Synthetic Construct
RSSQSIVHSTGNTYLE

SEQ ID NO: 5 - protein, 7 aa, Artificial Sequence
  Synthetic Construct
KVSNRFS

SEQ ID NO: 6 - protein, 9 aa, Artificial Sequence
  Synthetic Construct
FHGTHVPYT

SEQ ID NO: 7 - protein, 118 aa, Artificial Sequence
  Synthetic Construct
QVQLVQSGAEVKKPGSSVKVSCKASGYTFTDAYINWVRQAPGQGLEWMGWIWPGPVITYYNPKFKGRVTITADKSTSTAYMELSSLRSEDTAVYYCARREVLSPFAYWGQGTTVTVSS

SEQ ID NO: 8 - protein, 118 aa, Artificial Sequence
  Synthetic Construct
QVQLQQSGPELVKPGASVKISCKASGYTFTDAYINWVKQRPGQGLEWIGWIWPGPVITYYNPKFKGKATLTVDKSSSTAYMLLSSLTSEDSAFYFCARREVLSPFAYWGQGTLVTVSA

SEQ ID NO: 9 - protein, 112 aa, Artificial Sequence
  Synthetic Construct
DIVMTQSPDSLAVSLGERATINCRSSQSIVHSTGNTYLEWYQQKPGQPPKLLIYKVSNRFSGVPDRFSGSGSGTDFTLTISSLQAEDVAVYYCFHGTHVPYTFGGGTKVEIK

SEQ ID NO: 10 - protein, 112 aa, Artificial Sequence
  Synthetic Construct
DIQMTQSPSSLSASVGDRVTITCRSSQSIVHSTGNTYLEWYQQKPGKAPKLLIYKVSNRFSGVPSRFSGSGSGTDFTLTISSLQPEDFATYYCFHGTHVPYTFGGGTKVEIK

SEQ ID NO: 11 - protein, 112 aa, Artificial Sequence
  Synthetic Construct
DVLMTQTPLSLPVSLGDQASISCRSSQSIVHSTGNTYLEWYLQKPGQSPKLLIYKVSNRFSGVPDRFSGSGSGTDFTLKISRVEAEDLGVYYCFHGTHVPYTFGGGTKLEIK

SEQ ID NO: 12 - protein, 444 aa, Artificial Sequence
  Synthetic Construct
QVQLVQSGAEVKKPGSSVKVSCKASGYTFTDAYINWVRQAPGQGLEWMGWIWPGPVITYYNPKFKGRVTITADKSTSTAYMELSSLRSEDTAVYYCARREVLSPFAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 219 aa, Artificial Sequence
  Synthetic Construct
DIVMTQSPDSLAVSLGERATINCRSSQSIVHSTGNTYLEWYQQKPGQPPKLLIYKVSNRFSGVPDRFSGSGSGTDFTLTISSLQAEDVAVYYCFHGTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 219 aa, Artificial Sequence
  Synthetic Construct
DIQMTQSPSSLSASVGDRVTITCRSSQSIVHSTGNTYLEWYQQKPGKAPKLLIYKVSNRFSGVPSRFSGSGSGTDFTLTISSLQPEDFATYYCFHGTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 354 bp, Artificial Sequence
  Synthetic Construct
caggtgcagctggtgcagtctggggctgaggtgaagaagcctgggtcctcagtgaaggtttcctgcaaggcatctggctacaccttcactgacgcgtatataaactgggtgcgacaggcccctggacaagggcttgagtggatgggatggatttggcctggacccgttattacttactacaatccgaagttcaagggcagagtcaccattaccgcggacaaatccacgagcacagcctacatggagctgagcagcctgagatctgaggacacggccgtgtattactgtgcgagaagggaagtactatccccgtttgcttactggggccaaggaaccacggtcaccgtctcctca

SEQ ID NO: 16 - DNA, 336 bp, Artificial Sequence
  Synthetic Construct
gacatcgtgatgacccagtctccagactccctggctgtgtctctgggcgagagggccaccatcaactgcagatctagtcagagcattgtacatagtactggaaacacctatttagaatggtaccagcagaaaccaggacagcctcctaagctgctcatttacaaagtttccaaccgattttctggggtccctgaccgattcagtggcagcgggtctgggacagatttcactctcaccatcagcagcctgcaggctgaagatgtggcagtttattactgttttcacggcactcatgttccgtacacgttcggcggagggaccaaggtggagatcaaa

SEQ ID NO: 17 - DNA, 336 bp, Artificial Sequence
  Synthetic Construct
gacatccagatgacccagtctccatcctctctgtctgcatctgtaggagacagagtcaccatcacttgcagatctagtcagagcattgtacatagtactggaaacacctatttagaatggtatcagcagaaaccagggaaagcccctaagctcctgatctataaagtttccaaccgattttctggggtcccatcaaggttcagtggcagtggatctgggacagatttcactctcaccatcagcagtctgcaacctgaagattttgcaacttactactgttttcacggcactcatgttccgtacacgttcggcggagggaccaaggtggagatcaaa

SEQ ID NO: 18 - protein, 50 aa, Homo sapiens
VVSHFNDCPDSHTQFCFHGTCRFLVQEDKPACVCHSGYVGARCEHADLLA

SEQ ID NO: 19 - protein, 50 aa, Mus musculus
VVSHFNKCPDSHTQYCFHGTCRFLVQEEKPACVCHSGYVGVRCEHADLLA

SEQ ID NO: 20 - protein, 50 aa, Rattus norvegicus; identical to SEQ ID NO: 19

SEQ ID NO: 21 - protein, 50 aa, Macaca fascicularis; identical to SEQ ID NO: 18

SEQ ID NO: 22 - protein, 47 aa, Artificial Sequence
  Mature Human Epiregulin addition of N-terminal methionine
MVSITKCSSDMNGYCLHGQCIYLVDMSQNYCRCEVGYTGVRCEHFFL

SEQ ID NO: 23 - protein, 47 aa, Artificial Sequence
  Mature Mus musculus Epiregulin addition of N-terminal methionine
MVQITKCSSDMDGYCLHGQCIYLVDMREKFCRCEVGYTGLRCEHFFL

SEQ ID NO: 24 - protein, 46 aa, Macaca fascicularis
VSITKCNSDMNGYCLHGQCIYLVDMSQNYCRCEVGYTGVRCEHFYL

SEQ ID NO: 25 - protein, 72 aa, Homo sapiens
AVTVTPPITAQQADNIEGPIALKFSHLCLEDHNSYCINGACAFHHELEKAICRCFTGYTGERCEHLTLTSYA

SEQ ID NO: 26 - protein, 51 aa, Mus musculus
LKFSHPCLEDHNSYCINGACAFHHELKQAICRCFTGYTGQRCEHLTLTSYA

SEQ ID NO: 27 - protein, 54 aa, Artificial Sequence
  Mature Human EGF addition of N-terminal methionine
MNSDSECPLSHDGYCLHDGVCMYIEALDKYACNCVVGYIGERCQYRDLKWWELR

SEQ ID NO: 28 - protein, 86 aa, Homo sapiens
DLQEADLDLLRVTLSSKPQALATPNKEEHGKRKKKGKGLGKKRDPCLRKYKDFCIHGECKYVKELRAPSCICHPGYHGERCHGLSL

SEQ ID NO: 29 - protein, 80 aa, Homo sapiens
DGNSTRSPETNGLLCGDPEENCAATTTQSKRKGHFSRCPKQYKHYCIKGRCRFVVAEQTPSCVCDEGYIGARCERVDLFY

SEQ ID NO: 30 - protein, 98 aa, Homo sapiens
SVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSK

SEQ ID NO: 31 - protein, 442 aa, Artificial Sequence
  Synthetic Construct
QVQLQQSGPELVKPGASVKISCKASGYTFTDAYINWVKQRPGQGLEWIGWIWPGPVITYYNPKFKGKATLTVDKSSSTAYMLLSSLTSEDSAFYFCARREVLSPFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 219 aa, Artificial Sequence
  Synthetic Construct
DVLMTQTPLSLPVSLGDQASISCRSSQSIVHSTGNTYLEWYLQKPGQSPKLLIYKVSNRFSGVPDRFSGSGSGTDFTLKISRVEAEDLGVYYCFHGTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 33 - protein, 46 aa, Homo sapiens
VSITKCSSDMNGYCLHGQCIYLVDMSQNYCRCEVGYTGVRCEHFFL